(19) United States
(12) Patent Application Publication
(10) Pub. No.: US 2009/0076141 A1
(43) Pub. Date: Mar. 19, 2009

(54) Use of Propofol Prodrugs for Treating Neuropathic Pain

(75) Inventor: Peter A. Virsik, Portola Valley, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc., Santa Clara, CA (US)

(21) Appl. No.: 12/209,783

(22) Filed: Sep. 12, 2008

Related U.S. Application Data
(60) Provisional application No. 60/972,642, filed on Sep. 14, 2007.

(51) Int. Cl. A61K 31/265

(52) U.S. Cl. 514/512

(57) ABSTRACT

Methods of treating alcohol neuropathic pain in a patient comprising orally administering a therapeutically effective amount of a propofol prodrug having high oral bioavailability are disclosed.

This application claims the benefit under 35 U.S.C. § 119(e) of U.S. Provisional Application Ser. No. 60/972,642 filed Sep. 14, 2007, which is incorporated by reference in its entirety.

### FIELD

Disclosed herein are methods of treating neuropathic pain using propofol prodrugs having a high oral bioavailability.

### BACKGROUND

It is estimated that neuropathic pain affects over 6 million patients in the U.S. and Europe and over 26 million patients worldwide. Neuropathic pain involves an abnormal processing of sensory input usually occurring after direct injury or damage to nerve tissue. Neuropathic pain is a collection of disorders characterized by different etiologies including infection, inflammation, disease such as diabetes and multiple sclerosis, trauma or compression to major peripheral nerves, and chemical or irradiation-induced nerve damage (Jensen et al., Eur J Pharmacol 2001, 429, 1-11). Neuropathic pain typically persists long after tissue injury has resolved.

The role of N-methyl-D-aspartate (NMDA) receptors in the development and maintenance of chronic pain associated with central and peripheral nerve injury is well documented. Consequently, NMDA antagonists have been proposed as potential therapeutics for neuropathic pain. NMDA antagonists of different classes have shown efficacy in preclinical models as well as in patients with chronic pain, including neuropathic pain. However, large-scale clinical use of NMDA antagonists for the treatment of neuropathic pain is limited by unacceptable side effects (hallucinations, sedation, ataxia) of currently available compounds of this class. Several clinical studies have observed a long-lasting relief in some neuropathic pain patients treated with NMDA antagonists (Pud et al., Pain 1998, 75(2-3), 349-54; Eisenberg et al., J Pain 2007, 8(3), 223-9; and Rabben et al., J Pharmacol Exp Ther 1999, 289(2), 1060-1066.

Propofol (2,6-diisopropylphenol),

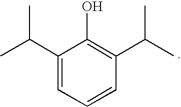

is a low molecular weight phenol that is widely used as an intravenous sedative-hypnotic agent in the induction and maintenance of anesthesia and/or sedation in mammals. The advantages of propofol as an anesthetic include rapid onset of anesthesia, rapid clearance, and minimal side effects (Langley et al., Drugs 1988, 35, 334-372). The hypnotic effects of propofol may be mediated through interaction with the GABA_{A} receptor complex, a hetero-oligomeric ligand-gated chloride ion channel (Peduto et al., Anesthesiology 1991, 75, 1000-1009). Propofol directly activates the GABA_{A} receptor chloride ionophore complex, increasing chloride conductance. In addition, propofol inhibits the NMDA subtype of glutamate receptor, possibly through an allosteric modulation of channel gating thereby depressing glutamate synaptic transmission. The action of propofol on GABA_{A} and NMDA receptors may explain the efficacy in refractory status epilepticus and delirium tremens. Other mechanisms and sites of action believed to contribute to the pharmacological activity of propofol include sensitivity to glycine receptors, inhibition of nicotinic receptor function (interactions with G-protein coupled receptors, and interactions with voltage-dependent sodium channels.

Another surgical anesthetic, ketamine, which is also an NMDA receptor antagonist, is shown effective in treating neuropathic pain (complex regional pain syndrome and post-herpetic neuralgia) in limited clinical studies (Harbut et al., US 2005/0148673). Ketamine, an anesthetic with a mechanism of action primarily as an NMDA receptor antagonist exhibits an anti-hyperalgesic, anti-allodynic, or tolerance-protective effect in patients at risk of pathological pain such as pain related to opioid tolerance, acute sever pain, neuropathic, ischemic, visceral, cancer or chronic post-surgical pain (CPSP) at sub-anesthetic doses. Low-dose intravenous ketamine may reduce peripheral neuropathic pain and spinal cord injury pain, fibromyalgia symptoms including tender point count and aerobic endurance, lower limb ischemic rest pain and chronic phantom limb pain (Visser and Schug, Biomedicine & Pharmacotherapy 2006, 60, 341-348; and Jorum et al., Pain 2003, 101, 229-235).

Propofol has been used to control cancer pain in patients (Hooke et al., J Ped Oncology Nursing 2007, 24(1), 29-34), and in pre-clinical studies, locally injected propofol produces an antinociceptive effect in an animal models of inflammatory pain (Guindon et al., Anesth Analg 2007, 104, 1563-1569). Propofol has also been shown to be effective in the treatment of central pain such as trigeminal neuralgia (Kubota et al., Exp Brain Res. 2007, 179(2), 181-190; and Mizuno et al., Neurol Med Chir (Tokyo) 2000, 40(7), 347-50), spinal cord injury (SCI) pain (Canavero and Bonicalzi, Neurol Sci 2001, 22, 271-273; and Canavero and Bonicalzi, Clin Neuropharmacol 2004, 27(4), 182-186), and central post-stroke pain (CPSP) (Canavero et al., J Neurol 1995, 242(9), 561-567; and Canavero and Bonicalzi, Pain 1998, 74(2-3), 109-114).

Propofol is rapidly metabolized in mammals with the drug being eliminated predominantly as glucuronidated and sulfated conjugates of propofol and 4-hydroxypropofol). Propofol is poorly absorbed in the gastrointestinal tract and only from the small intestine. When orally administered as a homogeneous liquid suspension, propofol exhibits an oral bioavailability of less than 5% that of an equivalent intravenous dose of propofol. Propofol clearance exceeds liver blood flow, which indicates that extrahepatic tissues contribute to the overall metabolism of the drug. Human intestinal mucosa glucuronidates propofol in vitro and oral dosing studies in rats indicate that approximately 90% of the administered drug undergoes first pass metabolism, with extraction by the intestinal mucosa accounting for the bulk of this pre-systemic elimination. Because of its poor oral bioavailability and extensive first-pass metabolism, propofol is administered by injection or intravenous infusion and oral administration of propofol has not been considered therapeutically effective. This has prevented investigations into the efficacy of propofol for treating pathologies and diseases or conditions for which intravenous infusion is not appropriate.

Recently, several methods for improving propofol absorption from the gastrointestinal tract and/or minimizing first pass metabolism have been demonstrated. For example, propofol prodrugs that exhibit enhanced oral bioavailability and that are sufficiently labile under physiological conditions to provide therapeutically effective concentrations of propofol following oral administration have been described by Gallop et al., U.S. Pat. No. 7,220,875, U.S. Pat. No. 7,230,003, and US 2006/0287525; and Xu et al., U.S. Pat. No. 7,241,807, US 2006/0100160, and US 2006/0205969, each of which is incorporated by reference herein in its entirety. These propofol prodrugs provide improved oral bioavailability of propofol and can also facilitate oral propofol regimens capable of providing therapeutically effective blood concentrations of propofol appropriate for treating chronic diseases and disorders. The use of such propofol prodrugs for treating emesis (Virsik and Xu, US 2007/0259933), for treating metabolic disease, cardiovascular disease, neurodegenerative disorders, liver disease and pulmonary disease (Virsik and Xu, US 2008/0161400), and for treating alcohol withdrawal, anxiety, central pain and pruritis (Cundy and Virsik, U.S. application Ser. No. 12/141,636 filed Jun. 8, 2008). Propofol prodrugs that provide a high oral bioavailability of propofol, such as the propofol prodrugs disclosed by Gallop et al. and by Xu et al., enable the use of orally administered propofol for treating neuropathic pain, potentially without the adverse effects associated with currently used pharmaceuticals.

### SUMMARY

Accordingly, methods of treating neuropathic pain in a patient are disclosed comprising orally administering to a patient in need of such treatment a therapeutically effective amount of a propofol prodrug that is capable of providing a high oral bioavailability of propofol. These and other features of the present disclosure are set forth herein.

### BRIEF DESCRIPTION OF THE DRAWINGS

The skilled artisan will understand that the drawings, described herein, are for illustration purposes only. The drawings are not intended to limit the scope of the present disclosure.

FIG. 1 shows propofol blood concentrations following oral administration of compound (2) to rats at doses from 25 mg-equivalent/kg to 300 mg-equivalent/kg of propofol.

FIG. 2 shows propofol blood concentrations following oral administration of compound (2) to rats at doses from 400 mg-equivalent/kg to 800 mg-equivalent/kg of propofol.

FIG. 3 shows propofol blood concentrations following oral administration of compound (2) to dogs at doses from 50 mg-equivalent/kg to 150 mg-equivalent/kg of propofol.

### DETAILED DESCRIPTION

### Definitions

A dash (“-”) that is not between two letters or symbols is used to indicate a point of attachment for a substituent. For example, —CONH₂ is attached through the carbon atom.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl(allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms (C₁₋₂₀) in certain embodiments, from 1 to 10 carbon atoms (C₁₋₁₀), in certain embodiments from 1 to 8 carbon atoms (C₁₋₈), in certain embodiments, from 1 to 6 carbon atoms (C₁₋₆), in certain embodiments from 1 to 4 carbon atoms (C₁₋₄), and in certain embodiments, from 1 to 3 carbon atoms (C₁₋₃).

“Acyl” by itself or as part of another substituent refers to a radical —C(O)R⁷⁰, where R⁷⁰ is hydrogen, alkyl, heteroalkyl, cycloalkyl, heterocycloalkyl, cycloalkylalkyl, heterocycloalkylalkyl, aryl, heteroaryl, arylalkyl, or heteroarylalkyl, which can be substituted, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR⁷¹ where R⁷¹ is alkyl, cycloalkyl, cycloalkylalkyl, aryl, or arylalkyl, which can be substituted, as defined herein. In some embodiments, alkoxy groups have from 1 to 8 carbon atoms. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR⁷² where R⁷² represents an alkyl, as defined herein. Examples of alkoxycarbonyl groups include, but are not limited to, methoxycarbonyl, ethoxycarbonyl, propoxycarbonyl, and butoxycarbonyl, and the like.

“Amino” refers to the radical —NH₂.

“Anesthesia” as used herein includes general anesthesia and deep sedation. General anesthesia is a drug-induced loss of consciousness during which patients are not arousable, even by painful stimulation. Deep sedation is a drug-induced depression of consciousness during which patients cannot be easily aroused but respond purposefully following repeated or painful stimulation. Reflex withdrawal from a painful stimulus is not a purposeful response. In deep sedation the ability of a patient to maintain ventilatory function may be impaired, while in general anesthesia, the ability to independently maintain ventilatory function is often impaired and often requires intervention in maintaining an open airway.

“Aryl” refers to a monovalent aromatic hydrocarbon radical derived by the removal of one hydrogen atom from a single carbon atom of a parent aromatic ring system. Aryl benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms chosen from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀). Aryl, however, does not encompass or overlap in any way with heteroaryl, separately defined herein.

“Arylalkyl” refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀.

“AUC” is the area under a curve representing the concentration of a compound in a biological fluid in a patient as a function of time following administration of the compound to the patient. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the disclosure herein, an AUC for propofol can be determined by measuring the concentration of propofol in the plasma or blood of a patient following oral administration of a dosage form comprising a propofol prodrug.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the highest drug concentration observed in the plasma or blood following a dose of drug.

“Carbamoyl” by itself or as part of another substituent refers to the radical —C(O)N(R¹⁸)R⁶⁹ where R⁶⁸ and R⁶⁹ are independently hydrogen, alkyl, substituted alkyl, aryl, substituted aryl, arylalkyl, substituted arylalkyl, heteroarylalkyl, substituted heteroarylalkyl, heteroaryl or substituted heteroaryl, as defined herein.

“Compounds” encompassed by structural Formulae (I)-(IV) disclosed herein include any specific compounds within these formulae. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

Compounds of Formulae (I)-(IV) include, but are not limited to, optical isomers of compounds of Formulae (I)-(IV), racemates thereof, and other mixtures thereof. In such embodiments, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formulae (I)-(IV) include Z- and E-forms (e.g., cis- and trans-forms) of compounds with double bonds.

In embodiments in which compounds of Formulae (I)-(IV) exist in various tautomeric forms, compounds of the present disclosure include all tautomeric forms of the compound. The compounds of Formulae (I)-(IV) may also exist in several tautomeric forms including the enol form, the keto form, and mixtures thereof. Accordingly, the chemical structures depicted herein encompass all possible tautomeric forms of the illustrated compounds. The compounds of Formulae (I)-(IV) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds as referred to herein include free acid, salt, solvates, hydrates and N-oxides forms of the compounds. Thus, when reference is made to compounds of the present disclosure, such as compounds of Formula (I)-(IV), it is understood that a compound also implicitly refers to salts, solvates, hydrates, and combinations of any of the foregoing. Certain compounds may exist in single or multiple crystalline or amorphous forms. In general, all physical forms are equivalent for the uses contemplated herein and are intended to be within the scope of the present disclosure.

Further, when partial structures of the compounds are illustrated, an asterisk (*) indicates the point of attachment of the partial structure to the rest of the molecule.

“Cycloalkoxycarbonyl” by itself or as part of another substituent refers to a radical —C(O)OR⁷⁶ where R⁷⁶ represents an cycloalkyl group as defined herein. Examples of cycloalkoxycarbonyl groups include, but are not limited to, cyclobutyloxycarbonyl, cyclohexyloxycarbonyl, and the like.

“Cycloalkyl” by itself or as part of another substituent refers to a partially saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₁₋₈ and the cycloalkyl moiety is C₃₋₁₂.

“Disease” refers to a disease, disorder, condition, or symptom of any of the foregoing.

“Dosage form” means a pharmaceutical composition in a medium, carrier, vehicle, or device suitable for administration to a patient.

“Halogen” refers to a fluoro, chloro, bromo, or iodo group. In certain embodiments, halogen refers to a chloro group.

“Heteroalkyl” by itself or as part of another substituent refer to an alkyl group in which one or more of the carbon atoms (and any associated hydrogen atoms) are independently replaced with the same or different heteroatomic groups. In some embodiments, heteroalkyl groups have from 1 to 8 carbon atoms. Examples of heteroatomic groups include, but are not limited to, —O, —S—, —O—O—, —S—S—, —O—S—, —NR⁷⁷R⁷⁸—, ═N—N═, —N═N—, —N═N—NR⁷⁹R⁸⁰, —PR⁸¹—, —P(O)₂—, —POR⁸²—, —O—P(O)₂—, —SO—, —SO₂—, —SnR⁸³R⁸⁴— and the like, where R⁷⁷, R⁷⁸, R⁷⁹, R⁸⁰, R⁸¹, R⁸², R⁸³, and R⁸⁴ are independently hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used. In certain embodiments, heteroatomic groups include, —O—, —S—, —O—O—, —S—S—, —O—S—, —NR⁸⁵, ═N—N═, —N═N—, —N═N—NR⁸⁵—, —PR⁸⁵, —P(O)₂—, —POR⁸⁵—, —O—P(O)₂—, —SO—, —SO₂—, —Sn(R⁸⁵)₂—, and the like, where each R³⁷ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatoms, groups having four carbon atoms and two heteroatoms, etc. In certain embodiments, each R¹⁰ is independently chosen from hydrogen and C₁₋₃ alkyl. In certain embodiments, a heteroatomic group is chosen from —O—, —S—, —NH—, —N(CH₃)—, and —SO₂—. In certain embodiments, a heteroatomic group is chosen from —O— and —NH—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl (C₆₋₃₀), e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Hydroxyl” refers to the group —OH.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutical composition” refers to at least one compound and a pharmaceutically acceptable vehicle with which the compound is administered to a patient.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound of Formulae (I)-(IV) may be administered to a patient and which does not destroy the pharmacological activity thereof, and which is nontoxic when administered in doses sufficient to provide a therapeutically effective amount of the compound.

“Prodrug” refers to a derivative of a drug molecule that requires a transformation within the body to release the active drug. Prodrugs are frequently, although not necessarily, pharmacologically inactive until converted to the parent drug. Prodrugs can be obtained by bonding a promoiety (defined herein) typically via a functional group, to a drug. For example, referring to compounds of Formulae (I)-(IV), the promoiety is bonded to propofol via the hydroxyl group of the drug.

“Prodrug of propofol” refers to a compound in which a promoiety that is cleavable in vivo, and is covalently bound to the propofol molecule. In certain embodiments, a prodrug may be actively transported by transporters expressed in the enterocytes lining the gastrointestinal tract such as, for example, the PEPT1 transporter. Propofol prodrugs can be stable in the gastrointestinal tract and following absorption are cleaved in the systemic circulation to release propofol. In certain embodiments, a prodrug of propofol provides a greater oral bioavailability of propofol compared to the oral bioavailability of propofol when administered as a uniform liquid immediate release formulation. In certain embodiments, a prodrug of propofol provides a high oral bioavailability of propofol, for example, exhibiting a propofol oral bioavailability that is at least 10 times greater than the oral bioavailability of propofol when orally administered in an equivalent dosage form. In certain embodiments, a prodrug of propofol is a compound having a structure encompassed by any one of Formulae (I)-(IV), compound (1), and/or compound (2), pharmaceutically acceptable salts thereof, or pharmaceutically acceptable solvates of any of the foregoing, infra. In certain embodiments, a propofol prodrug is compound (2), a pharmaceutically acceptable salt thereof, or a pharmaceutically acceptable solvate of any of the foregoing.

“Promoiety” refers to a chemical group, i.e. moiety, bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. Cleavage of the promoiety may proceed spontaneously, such as via a hydrolysis reaction, or may be catalyzed or induced by another agent, such as by an enzyme, by light, by acid, or by a change of or exposure to a physical or environmental parameter such as a change of temperature, pH, etc. The agent may be endogenous to the conditions of use, such as an enzyme present in the systemic circulation of a patient to which the prodrug is administered or the acidic conditions of the stomach, or the agent may be supplied exogenously. As an example, for a prodrug of Formula (IV), the promoiety is:

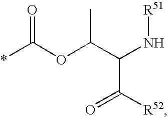

where R⁵¹ and R⁵² are as defined herein, and the drug is propofol.

“Sedation” as used herein refers to minimal sedation and/or moderate sedation (see e.g., American Society of Anesthesiologists, Anesthesiology 2002, 96, 1004-17). Minimal sedation, also referred to as anxiolysis, is a minimally depressed level of consciousness that retains the patient's ability to independently and continuously maintain an airway and respond appropriately to physical stimulation or verbal command that is produced by a pharmacological or non-pharmacological method or combination thereof. Although cognitive function and coordination may be modestly impaired, ventilatory and cardiovascular functions are unaffected. When the intent is minimal sedation in adults, the appropriate dosing is no more than the maximum recommended dose that can be prescribed for unmonitored home use, e.g., a maximum recommended therapeutic dose. Moderate sedation is a drug-induced depression of consciousness during which patients respond purposefully to verbal commands, either alone or accompanied by light tactile stimulation. No intervention is required to maintain a patient's airway. Sedation is a continuum and it is not always possible to predict how an individual patient will respond. A sedative dose can be determined by incremental dosing, administering multiple doses of a drug, such as a propofol prodrug provided by the present disclosure, until a desired effect is reached. A variety of scales can be used to assess sedation including, for example, the Ramsay scale, and the Observer's Assessment of Alertness/Sedation scale, and others. Objective measures of sedation include measurement of electroencephalogram parameters such as the Bispectral Index version XP and the Patient State Analyzer. In certain embodiments, sedation refers to minimal sedation, and in certain embodiments, moderate sedation.

“Solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water including monohydrates and hemi-hydrates.

“Substantially one diastereomer” refers to a compound containing two or more stereogenic centers such that the diastereomeric excess (d.e.) of the compound is greater than or about at least 90%. In certain embodiments, the d.e. is, for example, greater than or at least about 91%, about 92%, about 93%, about 94%, about 95%, about 96%, about 97%, about 98%, or about 99%.

“Substituted” refers to a group in which one or more hydrogen atoms are independently replaced with the same or different substituent(s). Examples of substituents include, but are not limited to, -Q, —R⁶⁰, —O⁻, —OH, ═O, —OR⁶⁰, —SR⁶⁰, —S⁻, ═S, —NR⁶⁰R⁶¹, ═NR⁶⁰, —CX₃, —CN, —CF₃, —OCN, —SCN, —NO, —NO₂, ═N₂, —N₃, —S(O)₂O⁻, —S(O)₂OH, —S(O)₂R⁶⁰, —OS(O₂)O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰ where each Q is independently a halogen; each R⁶⁰ and R⁶¹ are independently chosen from hydrogen, alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, and substituted heteroarylalkyl; or R⁶⁰ and R⁶¹ together with the nitrogen atom to which they are bonded form a ring chosen from a heterocycloalkyl, substituted heterocycloalkyl, heteroaryl, and substituted heteroaryl ring, and R⁶² and R⁶³ are independently chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, and substituted heteroarylalkyl, or R⁶² and R⁶³ together with the atom to which they are bonded form a ring chosen from a heterocycloalkyl, substituted heterocycloalkyl, heteroaryl, and substituted heteroaryl ring. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substituent groups: F, Cl, Br, C₁₋₃ alkyl, substituted alkyl, C₁₋₃ alkoxy, —S(O)₂NR⁶⁰R⁶¹, —NR⁶⁰R⁶¹, —CF₃, —OCF₃, —CN, —NR⁶⁰S(O)₂R⁶¹, —NR⁶⁰C(O)R⁶¹, C₅₋₁₀ aryl, substituted C₅₋₁₀ aryl, C₅₋₁₀ heteroaryl, substituted C₅₋₁₀ heteroaryl, —C(O)OR⁶⁰, —NO₂, —C(O)R⁶¹, —C(O)NR⁶⁰R⁶¹, —OCHF₂, C₁₋₃ acyl, —SR⁶⁰, —S(O)₂OH, —S(O)₂R⁶⁰, —S(O)R⁶⁰, —C(S)R⁶⁰, —C(O)O⁻, —C(S)OR⁶⁰, —NR⁶⁰C(O)NR⁶¹R⁶², —NR⁶⁰C(S)NR⁶¹R⁶², and —C(NR⁶⁰)NR⁶¹R⁶², C₃₋₈ cycloalkyl, and substituted C₃₋₈ cycloalkyl, wherein R⁶⁰, R⁶¹, and R⁶² are independently chosen from hydrogen and C₄ alkyl.

In certain embodiments, each substituent group can independently be chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

In certain embodiments, each substituent group is independently chosen from halogen, —OH, —CN, —CF₃, ═O, —NO₂, —C(O)NH₂, —R⁶⁰, —OR⁶⁰, —COOR⁶⁰, and —NR⁶⁰₂ wherein each R⁶⁰ is independently chosen from hydrogen and C₁₋₃ alkyl. In certain embodiments, each substituent group is independently chosen from halogen, —OH, —CN, —CF₃, —NO₂, —R⁶⁰, —OR⁶⁰, and —NR⁶⁰₂ wherein each R⁶⁰ is independently chosen from hydrogen and C₁₋₃ alkyl. In certain embodiments, each substituent group is independently chosen from halogen, —OH, —CN, —CF₃, ═O, —NO₂, —C(O)NR⁶⁰₂, —R⁶⁰, —OR⁶⁰, —COOR⁶⁰, and —NR⁶⁰₂ wherein each R⁶⁰ is independently chosen from hydrogen and C₁₋₆ alkyl. In certain embodiments, each substituent group is independently chosen from —OH, C₁₋₄ alkyl, and —NH₂.

“Controlled delivery” means continuous or discontinuous release of a compound over a prolonged period of time, wherein the compound is released at a controlled rate over a controlled period of time in a manner that provides for upper gastrointestinal and lower gastrointestinal tract delivery, coupled with improved compound absorption as compared to the absorption of the compound in an immediate release oral dosage form.

“Sustained release” refers to release of a therapeutic amount of a drug, a prodrug, or an active metabolite of a prodrug over a period of time that is longer than that of a conventional formulation of the drug, e.g. an immediate release formulation of the compound. For oral formulations, the term “sustained release” typically means release of the compound within the gastrointestinal tract lumen over a time period from about 2 to about 30 hours, and in certain embodiments, over a time period from about 4 to about 24 hours. Sustained release formulations achieve therapeutically effective concentrations of the drug in the systemic circulation over a prolonged period of time relative to that achieved by oral administration of an immediate release formulation of the drug. “Delayed release” refers to release of a drug, a prodrug, or an active metabolite of a prodrug into the gastrointestinal lumen after a delayed time period, for example a delay of about 1 to about 12 hours, relative to that achieved by oral administration of an immediate release formulation of the drug.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease, disorder, or at least one of the clinical symptoms of a disease or disorder. “Treating” or “treatment” also refers to inhibiting the disease, disorder, or at least one of the clinical symptoms of a disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” refers to a dose of a drug, prodrug or active metabolite of a prodrug that provides effective treatment of a disease or disorder in a patient. A therapeutically effective dose may vary from compound to compound and from patient to patient, and may depend upon factors such as the condition of the patient and the route of delivery. A therapeutically effective dose may be determined in accordance with routine pharmacological procedures known to those skilled in the art.

Reference is now made in detail to embodiments of the present disclosure. The disclosed embodiments are not intended to be limiting of the claims. To the contrary, the claims are intended to cover alternatives, modifications, and equivalents.

### Propofol Prodrugs

In certain embodiments, propofol prodrugs provide an oral bioavailability of propofol that is at least 10 times greater than the oral bioavailability of propofol when orally administered as an equivalent dose of propofol in an equivalent dosage form. In certain embodiments, propofol prodrugs provide an oral bioavailability of propofol that is at least 10 times greater than the oral bioavailability of propofol provided by an equivalent dose of propofol when orally administered to a patient as a uniform liquid immediate release formulation.

Propofol prodrugs include prodrugs, conjugates, and complexes in which propofol is attached to at least one moiety. The moiety covalently or non-covalently attached to propofol may enhance permeability through gastrointestinal epithelia via passive and/or active transport mechanisms, may control the release of propofol in the gastrointestinal tract, and/or may inhibit enzymatic and chemical degradation of propofol in the gastrointestinal tract. For propofol prodrugs in which the moiety remains attached to the propofol molecule after absorption, the moiety may enhance permeability through other biological membranes, and/or can inhibit enzymatic and chemical degradation of propofol in the systemic circulation.

Reducing the rate of metabolism of a drug in the gastrointestinal tract and/or enhancing the rate by which a drug is absorbed from the gastrointestinal tract may enhance the oral bioavailability of a drug. An orally administered drug will pass through the gastrointestinal system in about 11 to 31 hours. In general, an orally ingested drug resides about 1 to 6 hours in the stomach, about 2 to 7 hours in the small intestine, and about 8 to 18 hours in the colon. The oral bioavailability of a particular drug will depend on a number of factors including the residence time in a particular region of the gastrointestinal tract, the rate the drug is metabolized within the gastrointestinal tract, the rate at which a drug is metabolized in the systemic circulation, and the rate by which the compound is absorbed from a particular region or regions of the gastrointestinal tract, which include passive and active transport mechanisms. Several methods have been developed to achieve these objectives, including drug modification, incorporating the drug or modified drug in a controlled release dosage form, and/or by co-administering adjuvants, which can be incorporated in the dosage form containing the active compound.

Examples of propofol prodrugs that provide a high oral bioavailability of propofol include bile acid prodrugs, peptide conjugates, and prodrugs in which propofol is bonded to an amino acid or small peptide via a linkage. Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or to enhance absorption via active transport mechanisms, e.g., substrate promoieties. In particular, active transporters differentially expressed in regions of the gastrointestinal tract may be preferentially targeted to enhance absorption. For example, a propofol prodrug may incorporate a promoiety that is a substrate of the PEPT1 transporter expressed in the small intestine. Zerangue et al., U.S. Pat. No. 6,955,888 and US 2005/0214853, each of which is incorporated by reference herein in its entirety, disclose methodologies for screening drugs, conjugates or conjugate moieties, linked or linkable to drugs, for their capacity to be transported as substrates via the PEPT1 and PEPT2 transporters, which are known to be expressed in the human small intestine. Zerangue et al., US 2003/0158254 also disclose several transporters expressed in the human colon including the sodium dependent multi-vitamin transporter (SMVT) and monocarboxylate transporters MCT1 and MCT4, and methods of identifying agents, or conjugate moieties that are transporter substrates, and agents, conjugates, and conjugate moieties that may be screened for substrate activity. Zerangue et al. further disclose compounds that may be screened and are variants of known transporter substrates such as bile salts or acids, steroids, ecosanoids, or natural toxins or analogs thereof, as well as the linkage of drugs to conjugate moieties.

These prodrugs, which can provide enhanced oral bioavailability of propofol, are distinguishable from propofol prodrugs having promoieties that provide enhanced aqueous solubility of propofol for intravenous administration. Propofol exhibits poor aqueous solubility and it is desirable that intravenously administered drugs be water-soluble. Propofol is widely used as a hypnotic sedative for intravenous administration in the induction and maintenance of anesthesia or sedation in humans and animals. Propofol prodrugs with enhanced aqueous solubility for intravenous administration are disclosed, for example, by Stella et al., U.S. Pat. No. 6,204,257, U.S. Pat. No. 6,872,838, and U.S. Pat. No. 7,244,718; Marappan et al., U.S. Pat. No. 7,250,412; and Wingard et al., US 2005/0203068.

Examples of propofol prodrugs capable of providing an increased oral bioavailability of propofol in which propofol is bonded to an amino acid or small peptide via a linkage are disclosed in Gallop et al., U.S. Pat. No. 7,220,875, U.S. Pat. No. 7,230,003, and U.S. Pat. No. 7,230,003; Xu et al., U.S. Pat. No. 7,241,807; Xu et al., US 2006/0100160 and US 2006/0205969, each of which is incorporated by reference herein in its entirety.

In certain embodiments, prodrugs of propofol may be chosen from any of the genuses or species of compounds of Formula (I) as disclosed in Gallop et al., U.S. Pat. No. 7,220,875:

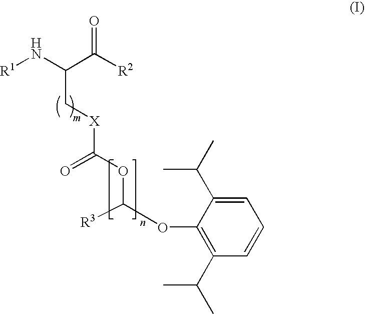

or a pharmaceutically acceptable salt thereof, wherein:

X is chosen from a bond, —CH₂—, —NR¹¹—, —O—, and —S—;

m is chosen from 1 and 2;

n is chosen from 0 and 1;

R¹ is chosen from hydrogen, [R⁵NH(CHR⁴)ₚC(O)]—, R⁶—, R⁶C(O)—, and R⁶OC(O)—;

R² is chosen from —OR⁷ and —[NR⁸(CHR⁹)_{q}C(O)OR⁷];

p and q are independently chosen from 1 and 2;

each R³ is independently chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;

each R⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁴ and R⁵ are attached to adjacent atoms then R⁴ and R⁵ together with the atoms to which they are bonded form a ring chosen from a heterocycloalkyl and substituted heterocycloalkyl ring;

R⁵ is chosen from hydrogen, R⁶, R⁶C(O)—, and R⁶OC(O)—;

R⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

each R⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁸ and R⁹ are attached to adjacent atoms then R⁸ and R⁹ together with the atoms to which they are bonded form a ring chosen from a heterocycloalkyl and substituted heterocycloalkyl ring; and

R¹¹ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

with the provisos that:
when R¹ is [R⁵NH(CHR⁴)PC(O)]— then R² is —OR⁷; and
when R² is —[NR⁸(CHR⁹)_{q}C(O)OR⁷] then R¹ is not [R⁵NH(CHR⁴)ₚC(O)]—.

In certain embodiment of a compound of Formula (I), n is 0.

In certain embodiment of a compound of Formula (I), the compound has Formula (Ia):

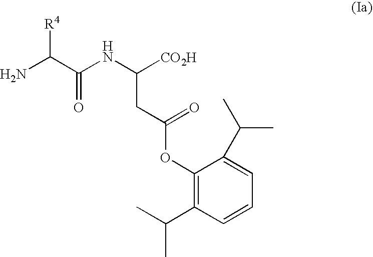

wherein R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (Ib):

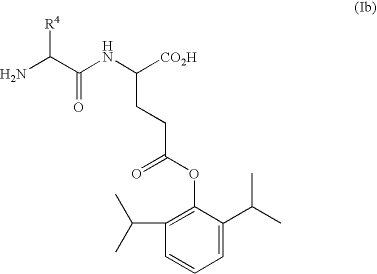

wherein R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (Ic):

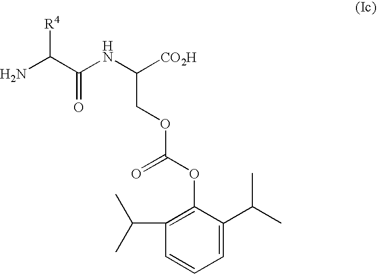

wherein R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (Id):

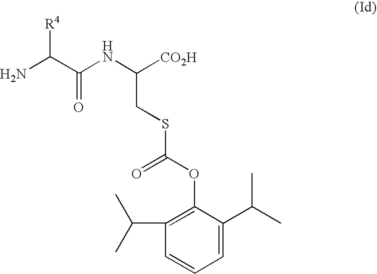

wherein R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (Ie):

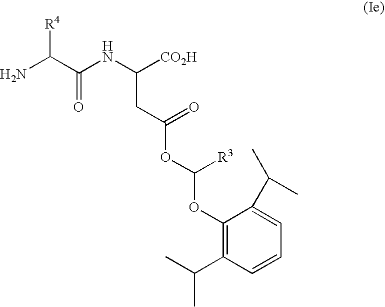

wherein R³ is hydrogen or methyl; and R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (If):

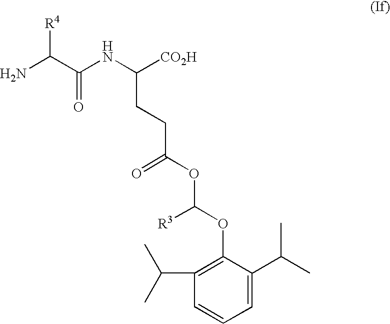

wherein R³ is hydrogen or methyl; and R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl.

In certain embodiment of a compound of Formula (I), the compound has Formula (Ig):

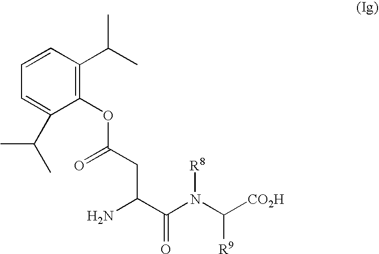

wherein R⁸ is hydrogen or methyl; and R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl, or R⁸ and R⁹ together with the atoms to which they are bonded form a cycloheteroalkyl or substituted cycloheteroalkyl ring.

In certain embodiment of a compound of Formula (Ig), R⁸ and R⁹ together with the atoms to which they are bonded form an azetidine, pyrrolidine or piperidine ring.

In certain embodiment of a compound of Formula (I), the compound has Formula (Ih):

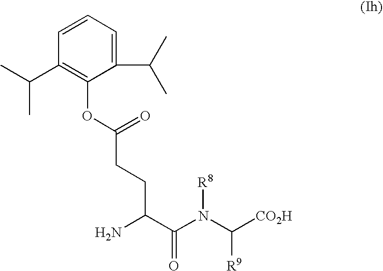

wherein R⁸ is hydrogen or methyl; and R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl; or R⁸ and R⁹ together with the atoms to which they are bonded form a cycloheteroalkyl or substituted cycloheteroalkyl ring.

In certain embodiment of a compound of Formula (Ih), R⁸ and R⁹ together with the atoms to which they are bonded form an azetidine, pyrrolidine or piperidine ring.

In certain embodiment of a compound of Formula (I), the compound has Formula (Ii):

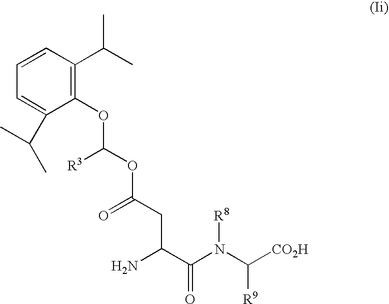

wherein R³ is hydrogen or methyl; R⁸ is hydrogen or methyl; and R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl; or R⁸ and R⁹ together with the atoms to which they are bonded form a cycloheteroalkyl or substituted cycloheteroalkyl ring.

In certain embodiment of a compound of Formula (Ii), R³ is hydrogen.

In certain embodiment of a compound of Formulae (Ia)-(Ii), R⁸ is hydrogen and R⁹ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Ii), R⁸ and R⁹ together with the atoms to which they are bonded form an azetidine, pyrrolidine or piperidine ring.

In certain embodiment of a compound of Formula (I), the compound has Formula (Ij):

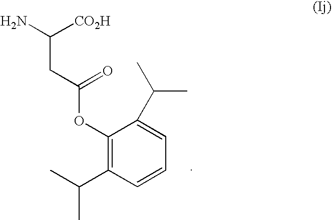

In certain embodiment of a compound of Formula (I), the compound has Formula (Ik):

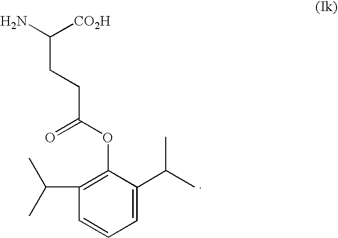

In certain embodiment of a compound of Formula (I), the compound has Formula (Il):

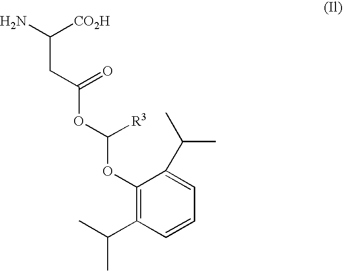

wherein R³ is hydrogen or methyl.

In certain embodiment of a compound of Formula (I), the compound has Formula (Im):

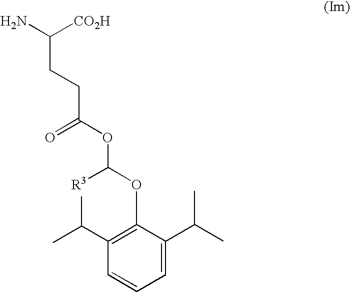

wherein R³ is hydrogen or methyl.

In certain embodiments, a compound provided by the present disclosure is a compound of Formula (In):

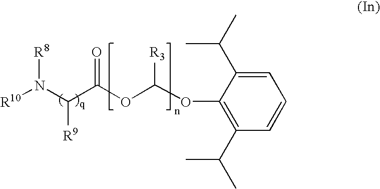

or a pharmaceutically acceptable salt, hydrate, solvate or N-oxide thereof, wherein:

n is 0 or 1;

R¹⁰ is hydrogen or [R⁵NH(CHR⁴)ₚC(O)]—;

p and q are independently 1 or 2;

R³ is chosen from the group consisting of hydrogen, alkyl, substituted alkyl, alkoxycarbonyl, aryl, substituted aryl, arylalkyl, carbamoyl, substituted carbamoyl, cycloalkyl, substituted cycloalkyl, cycloheteroalkyl, heteroaryl, substituted heteroaryl and heteroarylalkyl;

each R⁴ is independently chosen from the group consisting of hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl; or when R⁴ and R⁵ are attached to adjacent atoms then R⁴ and R⁵ together with the atoms to which they are bonded form a cycloheteroalkyl or substituted cycloheteroalkyl ring;

R⁵ is chosen from the group consisting of hydrogen, R⁶—, R⁶C(O)— and R⁶OC(O)—;

R⁶ is chosen from the group consisting of alkyl, substituted alkyl, aryl, substituted aryl, arylalkyl, substituted arylalkyl, cycloalkyl, substituted cycloalkyl, cycloheteroalkyl, heteroaryl, substituted heteroaryl and heteroarylalkyl;

R⁸ is chosen from the group consisting of hydrogen, alkyl, substituted alkyl, aryl, substituted aryl, arylalkyl, cycloalkyl, substituted cycloalkyl, cycloheteroalkyl, heteroaryl, substituted heteroaryl and heteroarylalkyl;

each R⁹ is independently chosen from the group consisting of hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl; or when R⁸ and R⁹ are attached to adjacent atoms then R⁸ and R⁹ together with the atoms to which they are bonded form a cycloheteroalkyl or substituted cycloheteroalkyl ring;

with the proviso that:

when R¹⁰ is hydrogen then n is 1.

In certain embodiment of a compound of Formula (In), n is 0.

In certain embodiment of a compound of Formula (In), the compound has Formula (Io):

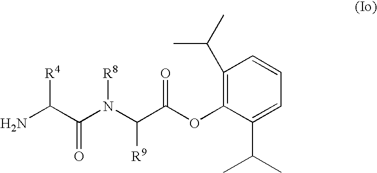

wherein R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl;

R⁸ is hydrogen or methyl; and

R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl; or R⁸ and R⁹ together with the atoms to which they are bonded form a cycloheteroalkyl or substituted cycloheteroalkyl ring.

In certain embodiment of a compound of Formula (Io), R⁸ is hydrogen and R⁹ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Io), R⁸ and R⁹ together with the atoms to which they are bonded form an azetidine, pyrrolidine or piperidine ring.

In certain embodiment of a compound of Formula (Io), R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Io), both the N- and C-terminal amino acid residues are of the L-configuration.

In certain embodiment of a compound of Formula (Io), R⁸ is hydrogen, R⁹ is methyl and R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Io), R⁸ is hydrogen, R⁹ is —CH₂CONH₂ and R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Io), R⁸ is hydrogen, R⁹ is benzyl and R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Io), R⁸ is hydrogen, R⁹ is 4-hydroxybenzyl and R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (In), the compound has Formula (Ip):

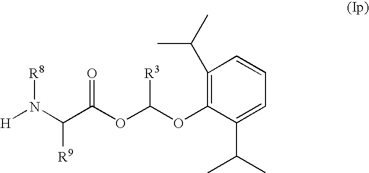

wherein R³ is hydrogen or methyl;

R⁸ is hydrogen or methyl; and

R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl; or R⁸ and R⁹ together with the atoms to which they are bonded form a cycloheteroalkyl or substituted cycloheteroalkyl ring.

In certain embodiment of a compound of Formula (Ip), R⁸ is hydrogen and R⁹ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Ip), R⁸ and R⁹ together with the atoms to which they are bonded form an azetidine, pyrrolidine or piperidine ring.

In certain embodiment of a compound of Formula (In), the compound of has Formula (Iq):

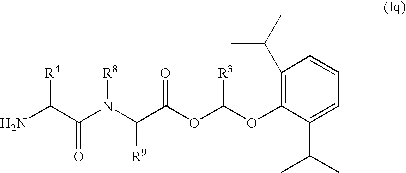

wherein R³ is hydrogen or methyl;

R⁴ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl;

R⁸ is hydrogen or methyl; and

R⁹ is chosen from the group consisting of hydrogen, alkanyl, substituted alkanyl, aryl, substituted aryl, arylalkanyl, substituted arylalkanyl, cycloalkanyl, heteroarylalkanyl and substituted heteroarylalkanyl, or R⁸ and R⁹ together with the atoms to which they are bonded form a cycloheteroalkyl or substituted cycloheteroalkyl ring.

In certain embodiment of a compound of Formula (Iq), R⁸ is hydrogen and R⁹ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiment of a compound of Formula (Iq), R⁸ and R⁹ together with the atoms to which they are bonded form an azetidine, pyrrolidine or piperidine ring.

In certain embodiment of a compound of Formula (Iq), R⁴ is chosen from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiments, prodrugs of propofol may be chosen from any of the genuses or species of compounds of Formula (II) as disclosed in Gallop et al., U.S. Pat. No. 7,230,003:

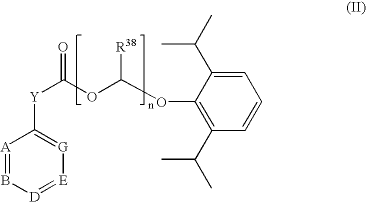

or a pharmaceutically acceptable salt thereof, wherein:

n is chosen from 0 and 1;

Y is chosen from a bond, CR²¹R²², NR²³, O, and S;

A is chosen from CR²⁴ and N;

B is chosen from CR²⁵ and N;

D is chosen from CR²⁶ and N;

E is chosen from CR²⁷ and N;

G is chosen from CR²⁸ and N;

R³⁸ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;

R²¹ and R²² are independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

R²³ is chosen from hydrogen, alkyl, substituted alkyl, aryl, arylalkyl, cycloalkyl, and heteroaryl;

R²⁴ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁵ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁶ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁷ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁸ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

W is chosen from a bond, —CR³²R³³, —NR³⁴, O, and S;

Z is chosen from —CR³⁵R³⁶, —NR³⁷, O, and S;

k is chosen from 0 and 1;

r is chosen from 1, 2, and 3;

each of R²⁹, R³⁰, R³¹, R³², R³³, R³⁵ and R³⁶ is independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and

R³⁴ and R³⁷ are independently chosen from hydrogen, alkyl, substituted alkyl, aryl, arylalkyl, cycloalkyl, and heteroaryl;

with the provisos that:
at least one of A, B, D, E, and G is not N;
one and only one of R²⁴, R²⁵, R²⁶, R²⁷, or R²⁸ is —W[C(O)]ₖZ(CR²⁹R³⁰)ᵣCO₂R³¹; and
if k is 0 then W is a bond.

In certain embodiments, prodrugs of propofol may be chosen from any of the genuses or species of compounds of Formula (III) as disclosed in Xu et al., U.S. Pat. No. 7,241,807:

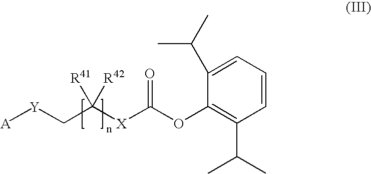

or a pharmaceutically acceptable salt thereof, wherein:

each R⁴¹ and R⁴² is independently chosen from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁴¹ and R⁴² together with the carbon atom to which they are bonded form a ring chosen from a cycloalkyl, substituted cycloalkyl, heterocycloalkyl, and substituted heterocycloalkyl ring;

A is chosen from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; or A, Y, and one of R⁴¹ and R⁴² together with the atoms to which they are bonded form a ring chosen from a heterocycloalkyl and substituted heterocycloalkyl ring;

Y is chosen from —O and —NR⁴³—;

R⁴³ is chosen from hydrogen, alkyl, substituted alkyl, arylalkyl, and substituted arylalkyl;

n is an integer from 1 to 5;

X is chosen from —NR⁴⁴—, —O, —CH₂, and —S—; and

R⁴⁴ is chosen from hydrogen, alkyl, substituted alkyl, arylalkyl, and substituted arylalkyl.

In certain embodiments, prodrugs of propofol may be chosen from any of the genuses or species of compounds of Formula (IV) as disclosed in Xu et al., US 2006/0100160:

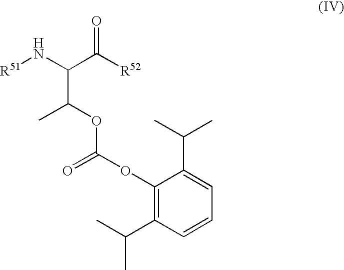

or a pharmaceutically acceptable salt thereof, wherein:

R⁵¹ is chosen from hydrogen, [R⁵⁵NH(CHR⁵⁴)ₚC(O)]—, R⁵⁶—, R⁵⁶C(O)—, and R⁵⁶OC(O)—;

R⁵² is chosen from —OR⁵⁷ and —[NR⁵⁸(CHR⁵⁹)_{q}C(O)OR⁵⁷];

p and q are independently chosen from 1 and 2;

each R⁵⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁵⁴ and R⁵⁵ are bonded to adjacent atoms then R⁵⁴ and R⁵⁵ together with the atoms to which they are bonded form a ring chosen from a heterocycloalkyl and substituted heterocycloalkyl ring;

R⁵⁵ is chosen from hydrogen, R⁵—, R⁵⁶C(O)—, and R⁵⁶OC(O)—;

R⁵⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁵⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁵⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and

each R⁵⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁵⁸ and R⁵⁹ are bonded to adjacent atoms then R⁵⁸ and R⁵⁹ together with the atoms to which they are bonded form a ring chosen from a heterocycloalkyl and substituted heterocycloalkyl ring;

with the proviso that when R⁵² is —[NR⁵⁸(CHR⁵⁹)_{q}C(O)OR⁵⁷] then R⁵¹ is not [R⁵⁵NH(CHR⁵⁴)ₚC(O)]—.

In certain embodiments of a compound of Formula (IV), the compound has Formula (IVa):

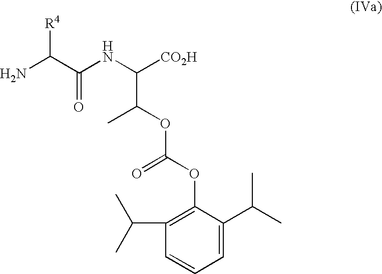

or a pharmaceutically acceptable salt thereof, wherein R⁴ is hydrogen, alkanyl, substituted alkanyl, aryl, substituted aryl, arylalkanyl, substituted arylalkanyl, cycloalkanyl, heteroarylalkanyl or substituted heteroarylalkanyl.

In certain embodiments of a compound of Formula (IVa), R⁴ is selected from the group consisting of hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂, —CH₂CH₂CH₂NHC(NH)NH₂, phenyl, benzyl, 4-hydroxybenzyl, 4-imidazolylmethyl and 3-indolylmethyl.

In certain embodiments of a compound of Formula (IVa), the N-terminal amino acid residue is of the L-configuration.

In certain embodiments of a compound of Formula (IVa), the N-terminal amino acid residue is of the D-configuration.

In certain embodiments of a compound of Formula (IVa), the C-terminal amino acid residue is of the L-configuration.

In certain embodiments of a compound of Formula (IVa), the C-terminal amino acid residue is of the D-configuration.

In certain embodiments of a compound of Formula (IV), the compound has Formula (IVb):

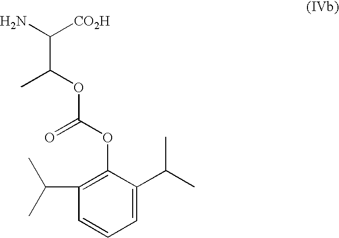

or a pharmaceutically acceptable salt thereof.

In certain embodiments of a compound of Formula (IVb), the amino acid residue is of the L-configuration.

In certain embodiments of a compound of Formula (IVb), the amino acid residue is of the D-configuration.

In certain embodiments, a prodrug of propofol is 2-amino-3-methyl-3-(2,6-diisopropyl-phenoxycarbonyloxy)-propanoic acid (compound (1)):

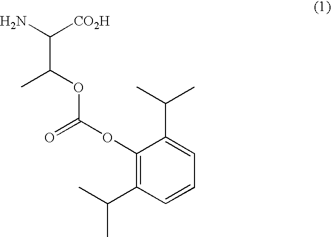

or a pharmaceutically acceptable salt thereof.

In certain embodiments of compound (1), the α-carbon of the amino acid residue is of the L-configuration. In certain embodiments of compound (1), the α-carbon of the amino acid residue is of the D-configuration.

In certain embodiments, a prodrug of propofol is 2-amino-3-(2,6-diisopropyl-phenoxycarbonyloxy)-propanoic acid (compound (2)) as disclosed in Xu et al., US 2006/0205969:

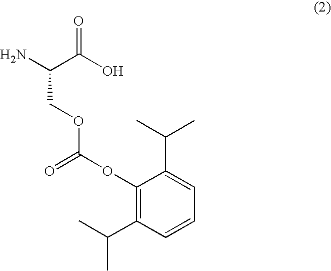

or a pharmaceutically acceptable salt thereof.

In certain embodiments, compound (2) is a crystalline form of 2-amino-3-(2,6-diisopropyl-phenoxycarbonyloxy)-propanoic acid, a pharmaceutically acceptable salt thereof, or a pharmaceutically acceptable solvate of any of the foregoing. In certain embodiments, a prodrug of propofol of Formula (2) may be a crystalline form of (S)-2-amino-3-(2,6-diisopropylphenoxycarbonyloxy)-propanoic, a pharmaceutically acceptable salt thereof, or a pharmaceutically acceptable solvate of any of the foregoing. In certain embodiments, a prodrug of propofol is crystalline 2-amino-3-(2,6-diisopropylphenoxycarbonyloxy)-propanoic acid hydrochloride. In certain embodiments, a prodrug of propofol is crystalline (S)-2-amino-3-(2,6-diisopropylphenoxycarbonyloxy)-propanoic acid hydrochloride. In certain embodiments, a prodrug of propofol is crystalline (S)-2-amino-3-(2,6-diisopropylphenoxy-carbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 19.4°±0.2°, 20.1°±0.2°, 21.3°±0.2°, 21.7°±0.2°, 22.5°±0.2°, 23.5°±0.2°, 24.4°±0.2°, 25.1°±0.2°, 26.8°±0.2°, 27.3°±0.2°, 27.8°±0.2°, 29.2°±0.2°, 29.6°±0.2°, 30.4°±0.2° and 33.4°±0.2° in an X-ray powder diffraction pattern measured using CuKα radiation. In certain embodiments, a prodrug of propofol is crystalline (S)-2-amino-3-(2,6-diisopropylphenoxycarbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 20.1°±0.2°, 22.5°±0.2°, 23.5°±0.2°, 25.1°±0.2°, 29.2°±0.2°, 29.6°±0.2°, and 33.4°±0.2° in an X-ray powder diffraction pattern measured using CuKα radiation.

In certain embodiments, a prodrug of propofol is crystalline 2-amino-3-(2,6-diisopropylphenoxycarbonyloxy)-propanoic acid hydrochloride having a melting point from about 180° C. to about 200° C. In certain embodiments, a prodrug of propofol is crystalline 2-amino-3-(2,6-diisopropylphenoxycarbonyloxy)-propanoic acid hydrochloride having a melting point from about 185° C. to about 195° C. In certain embodiments, a prodrug of propofol is crystalline (S)-2-amino-3-(2,6-diisopropylphenoxycarbonyloxy)-propanoic acid hydrochloride having a melting point from about 188° C. to about 189° C.

In certain embodiments, a prodrug of propofol is crystalline 2-amino-3-(2,6-diisopropylphenoxycarbonyloxy)-propanoic acid mesylate. In certain embodiments, a prodrug of propofol is crystalline (S)-2-amino-3-(2,6-diisopropylphenoxycarbonyloxy)-propanoic acid mesylate. In certain embodiments, a prodrug of propofol is crystalline (S)-2-amino-3-(2,6-diisopropylphenoxycarbonyloxy)-propanoic acid mesylate having characteristic peaks (2θ) at 4.2°±0.1°, 11.7°±0.1°, 12.1°±0.1°, 12.6°±0.1°, 16.8°±0.1°, 18.4°±0.2°, 21.0°±0.1°, 22.3°±0.1°, 22.8°±0.2°, 24.9°±0.2°, 25.3°±0.1°, 26.7°±0.2°, and 29.6°±0.1° in an X-ray powder diffraction pattern measured using CuKα radiation. In certain embodiments, a prodrug of propofol may be crystalline (S)-2-amino-3-(2,6-diisopropylphenoxycarbonyloxy)-propanoic acid mesylate having characteristic peaks (2η) at 4.2°±0.1°, 12.6°±0.1°, 16.8°±0.1°, 21.0°±0.1°, 25.3°±0.1°, 2 and 29.6°±0.1° in an X-ray powder diffraction pattern measured using CuKα radiation.

In certain embodiments, a prodrug of propofol is crystalline 2-amino-3-(2,6-diisopropylphenoxycarbonyloxy)-propanoic acid mesylate having a melting point from about 156° C. to about 176° C. In certain embodiments, a prodrug of propofol is crystalline 2-amino-3-(2,6-diisopropylphenoxycarbonyloxy)-propanoic acid mesylate having a melting point from about 161° C. to about 172° C. In certain embodiments, a prodrug of propofol is crystalline (S)-2-amino-3-(2,6-diisopropylphenoxycarbonyloxy)-propanoic acid mesylate having a melting point from about 166° C. to about 167° C.

Propofol prodrugs of Formulae (I)-(IV) may be administered orally and transported across cells (i.e., enterocytes) lining the lumen of the gastrointestinal tract. Certain of the compounds of structural Formulae (I)-(IV) may be substrates for the proton-coupled intestinal peptide transport system (PEPT1) (Leibach et al., Annu. Rev. Nutr. 1996, 16, 99-119), which mediates the cellular uptake of small intact peptides consisting of two or three amino acids that are derived from the digestion of dietary proteins. In the intestine, where small peptides are not effectively absorbed by passive diffusion, PEPT1 may act as a vehicle for the effective uptake of small peptides across the apical membrane of the gastric mucosa including propofol prodrugs of Formulae (I)-(IV).

Methods for determining whether propofol prodrugs of Formulae (I)-(IV) serve as substrates for the PEPT1 transporter are disclosed, for example, in Xu et al., US 2006/0100160. In vitro systems using cells engineered to heterologously express the PEPT1 transport system or cell-lines that endogenously express the transporter (e.g. Caco-2 cells) may be used to assay transport of compounds of Formulae (I)-(IV) by the PEPT1 transporter. Standard methods for evaluating the enzymatic conversion of a propofol prodrug to propofol in vitro are disclosed, for example, in Xu et al., US 2006/0100160.

Oral administration of propofol prodrugs to animals is described in Xu et al., U.S. Pat. No. 7,241,807, US 2006/0100160, and US 2006/0205969, and illustrate that propofol prodrugs can afford significant enhancement in the oral bioavailability of propofol relative to the oral bioavailability of propofol when administered in an equivalent dosage form. In certain embodiments, a prodrug of propofol provides greater than 10% absolute oral bioavailability of propofol, i.e., relative to the bioavailability of propofol following intravenous administration of an equimolar dose of propofol itself. In certain embodiments, a prodrug of propofol provides at least about 10 times higher oral bioavailability of propofol compared to the oral bioavailability of propofol itself, and in certain embodiments, at least about 40 times higher oral bioavailability of propofol compared to the oral bioavailability of propofol itself when orally administered in an equivalent dosage form (see, e.g., Xu et al., U.S. Pat. No. 7,241,807, US 2006/0100160, and US 2006/0205969).

Methods of synthesizing prodrugs of propofol of Formula (I) are disclosed in Gallop et al., U.S. Pat. No. 7,220,875. Methods of synthesizing prodrugs of propofol of Formula (II) are disclosed in Gallop et al., U.S. Pat. No. 7,230,003. Methods of synthesizing prodrugs of propofol of Formulae (III) are disclosed in Xu et al., U.S. Pat. No. 7,241,807. Methods of synthesizing prodrugs of propofol of Formulae (IV) are disclosed in Xu et al US 2006/0100160. Methods of synthesizing and crystallizing prodrugs of propofol of Formula (2) are disclosed in Xu et al., US 2006/0205969.

Propofol prodrugs of Formulae (I)-(IV) are distinguished from other propofol prodrugs by their ability to provide high oral bioavailability of propofol. Various prodrugs of propofol have been developed that enhance the aqueous solubility of propofol for intravenous administration (Stella et al., U.S. Pat. Nos. 6,204,257 and 6,872,838; Hendler et al., U.S. Pat. No. 6,254,853 and U.S. Pat. No. 6,362,234; Jenkins et al., U.S. Pat. No. 6,815,555; Wingard et al., US 2005/0203068; Marappan et al., U.S. Pat. No. 7,250,412; Orlando et al., US 2005/0267169; Fechner et al., Anesthesiology 2003, 99, 303-313; Fechner et al., Anesthesiology 2004, 101, 626-639; Struys et al., Anesthesiology 2005, 103, 730-43; and Gibiansky et al., Anesthesiology 2005, 103, 718-729). It has recently been shown that O-phosphonooxymethyl propofol exhibits a propofol oral bioavailability in rats of 22.2% relative to an equivalent invtravenous dose of propofol (Slusher and Wozniak, US 2007/0202158). However, the pharmacokinetic profile is characterized by a short time to maximum propofol concentration of about 30 minutes. Other analogs or derivatives of propofol are also known (Chandran US 2006/0241017 and US 2006/0287244; Tao et al., US 2007/0185217; and Desai et al., WO 2006/089120).

Any of the propofol prodrugs disclosed herein may exhibit sufficient stability to enzymatic and/or chemical degradation in the gastrointestinal tract resulting in enhanced oral bioavailability of the propofol prodrug and/or propofol metabolite. The propofol prodrugs may also exhibit enhanced passive and/or active gastrointestinal absorption compared to propofol.

### Pharmaceutical Compositions

Propofol prodrugs provided by the present disclosure may be formulated into pharmaceutical compositions for use in oral dosage forms to be administered to patients.

Pharmaceutical compositions comprise at least one propofol prodrug and at least one pharmaceutically acceptable vehicle. A pharmaceutical composition may comprise a therapeutically effective amount of at least one propofol prodrug and at least one pharmaceutically acceptable vehicle. Pharmaceutically acceptable vehicles include diluents, adjuvants, excipients, and carriers. Pharmaceutical compositions may be produced using standard procedures known in the art. Pharmaceutical compositions may take any form appropriate for oral delivery such as solutions, suspensions, emulsions, tablets, pills, pellets, granules, capsules, capsules containing liquids, powders, and the like. Pharmaceutical compositions provided by the present disclosure may be formulated so as to provide immediate, sustained, or delayed release of a propofol prodrug after administration to a patient by employing procedures known in the art.

Pharmaceutical compositions may include an adjuvant that facilitates absorption of a propofol prodrug through the gastrointestinal epithelia. Such absorption enhancers may, for example, open the tight-junctions in the gastrointestinal tract or modify the effect of cellular components, such as p-glycoprotein. Suitable enhancers include alkali metal salts of salicylic acid, such as sodium salicylate, caprylic, or capric acid, such as sodium caprylate or sodium caprate, sodium deoxycholate, and the like. Other adjuvants that enhance permeability of cellular membranes include resorcinol, surfactants, polyethylene glycol, and bile acids. Adjuvants may also reduce enzymatic degradation of a compound of a propofol pro drug. Microencapsulation using protenoid microspheres, liposomes, or polysaccharides may also be effective in reducing enzymatic degradation of administered compounds.

Propofol prodrugs provided by the present disclosure may be formulated in unit oral dosage forms. Unit oral dosage form refers to physically discrete units suitable for dosing to a patient undergoing treatment, with each unit containing a predetermined quantity of a propofol prodrug. Oral dosage forms comprising at least one propofol prodrug may be administered to patients as a dose, with each dose comprising one or more oral dosage forms. A dose may be administered once a day, twice a day, or more than twice a day, such as three or four times per day. A dose may be administered at a single point in time or during a time interval. Oral dosage forms comprising at least one propofol prodrug may be administered alone or in combination with other drugs for treating the same or different disease, and may continue as long as required for effective treatment of the disease. Oral dosage forms comprising a propofol prodrug may provide a concentration of propofol in the plasma, blood, or tissue of a patient over time, following oral administration of the dosage form to the patient. The propofol concentration profile may exhibit an AUC that is proportional to the dose of the propofol prodrug.

A dose comprises an amount of a propofol prodrug calculated to produce an intended therapeutic effect. An appropriate amount of a propofol prodrug to produce an intended therapeutic effect will depend, in part, on the oral bioavailability of propofol provided by the propofol prodrug, by the pharmacokinetics of the propofol prodrug, and by the properties of the dosage form used to administer the propofol prodrug. A therapeutically effective dose of a propofol prodrug may comprise from about 10 mg-equivalents to about 5,000 mg-equivalents of propofol, from about 50 mg-equivalents to about 2,000 mg-equivalents of propofol, and in certain embodiments, from about 100 mg-equivalents to about 1,000 mg-equivalents of propofol. In certain embodiments, a therapeutically effective dose of a propofol prodrug provides a blood concentration of propofol from about 10 ng/mL to about 5,000 ng/mL, in certain embodiments from about 100 ng/mL to about 2,000 ng/mL, and in certain embodiments from about 200 ng/mL to about 1,000 ng/mL for a continuous period of time following oral administration of a dosage form comprising a propofol prodrug to a patient. In certain embodiments, a therapeutically effective dose of a propofol prodrug provides a blood concentration of propofol that is therapeutically effective for treating a disease in a patient, and that is less than a concentration effective in causing sedation in the patient, for example, less than about 1,500 ng/mL or less than about 2,000 ng/mL. In certain embodiments, a therapeutically effective dose of a propofol prodrug provides a blood concentration of propofol that is therapeutically effective and that is less than a concentration effective for the maintenance of general anesthesia (e.g., a sub-hypnotic concentration), for example, less than about 3,000 ng/mL or less than about 10,000 ng/mL.

Oral dosage forms comprising a propofol prodrug may have immediate release or controlled release characteristics. Immediate release oral dosage forms release the propofol prodrug from the dosage form within about 30 minutes following ingestion. In certain embodiments, an oral dosage form provided by the present disclosure may be a controlled release dosage form. Controlled delivery technologies may improve the absorption of a drug in a particular region or regions of the gastrointestinal tract. Controlled drug delivery systems may be designed to deliver a drug in such a way that the drug level is maintained within a therapeutically effective blood concentration range for a period as long as the system continues to deliver the drug at a particular rate. Controlled drug delivery may produce substantially constant blood levels of a drug as compared to fluctuations observed with immediate release dosage forms. For some diseases maintaining a controlled concentration of propofol in the blood or in a tissue throughout the course of therapy is desirable. Immediate release dosage forms may cause blood levels to peak above the level required to elicit the desired response, which may cause or exacerbate side effects. Controlled drug delivery may result in optimum therapy, reduce the frequency of dosing, and reduce the occurrence, frequency, and/or severity of side effects. Examples of controlled release dosage forms include dissolution controlled systems, diffusion controlled systems, ion exchange resins, osmotically controlled systems, erodible matrix systems, pH independent formulations, gastric retention systems, and the like.

The appropriate oral dosage form for a particular propofol prodrug may depend, at least in part, on the gastrointestinal absorption properties of the propofol prodrug, the stability of the propofol prodrug in the gastrointestinal tract, the pharmacokinetics of the propofol prodrug, and the intended therapeutic profile of propofol. An appropriate controlled release oral dosage form may be selected for a particular propofol prodrug. For example, gastric retention oral dosage forms may be appropriate for propofol prodrugs absorbed primarily from the upper gastrointestinal tract, and sustained release oral dosage forms may be appropriate for propofol prodrugs absorbed primarily form the lower gastrointestinal tract.

Gastric retention dosage forms, i.e., dosage forms designed to be retained in the stomach for a prolonged period of time, can increase the bioavailability of drugs that are most readily absorbed from the upper gastrointestinal tract. The residence time of a conventional dosage form in the stomach is 1 to 3 hours. After transiting the stomach, there is approximately a 3 to 5 hour window of bioavailability before the dosage form reaches the colon. However, if the dosage form is retained in the stomach, the drug can be released before it reaches the small intestine and will enter the intestine in solution in a state in which it can be more readily absorbed. Another use of gastric retention dosage forms is to improve the bioavailability of a drug that is unstable to the basic conditions of the intestine. To enhance drug absorption from the upper gastrointestinal tract, several gastric retention dosage forms have been developed. Examples include hydrogels, buoyant matrices, polymer sheets, microcellular foams, and swellable dosage forms.

In swelling and expanding systems, dosage forms that swell and change density in relation to the surrounding gastric content may be retained in the stomach for longer than conventional dosage forms. Dosage forms can absorb water and swell to form a gelatinous outside surface and float on the surface of gastric content surface while maintaining integrity before releasing a drug. Fatty materials may be added to impede wetting and enhance flotation when hydration and swelling alone are insufficient. Materials that release gases may also be incorporated to reduce the density of a gastric retention dosage form. Swelling also may significantly increase the size of a dosage form and thereby impede discharge of the non-disintegrated swollen solid dosage form through the pylorus into the small intestine. Swellable dosage forms may be formed by encapsulating a core containing drug and a swelling agent, or by combining a drug, swelling agent, and one or more erodible polymers.

Gastric retention dosage forms may also be in the form of folded thin sheets containing a drug and water-insoluble diffusible polymer that opens in the stomach to its original size and shape so as to be sufficiently large to prevent or inhibit passage of the expanded dosage form through the pyloric sphincter.

Floating and buoyancy gastric retention dosage forms are designed to trap gases within sealed encapsulated cores that can float on the gastric contents, and thereby be retained in the stomach for a longer time, e.g., 9 to 12 hours. Due to the buoyancy effect, these systems provide a protective layer preventing the reflux of gastric content into the esophageal region and may also be used for controlled release devices. A floating system may, for example, contain hollow cores containing drug coated with a protective membrane. The trapped air in the cores floats the dosage form on the gastric content until the soluble ingredients are released and the system collapses. In other floating systems, cores comprise drug and chemical substances capable of generating gases when activated. For example, coated cores, comprising carbonate and/or bicarbonate generate carbon dioxide in the reaction with hydrochloric acid in the stomach or incorporated organic acid in the system. The gas generated by the reaction is retained to float the dosage form. The inflated dosage form later collapses and clears from the stomach when the generated gas permeates slowly through the protective coating.

Bioadhesive polymers may also provide vehicles for controlled delivery of drugs to a number of mucosal surfaces in addition to the gastric mucosa. Bioadhesive systems can be designed by incorporation of a drug and other excipients within a bioadhesive polymer. On ingestion, the polymer hydrates and adheres to the mucus membrane of the gastrointestinal tract. Bioadhesive polymers may be selected that adhere to a desired region or regions of the gastrointestinal tract. Bioadhesive polymers may be selected to optimized delivery to targeted regions of the gastrointestinal tract including the stomach and small intestine. The mechanism of the adhesion is thought to be through the formation of electrostatic and hydrogen bonding at the polymer-mucus boundary. Bioadhesive delivery systems useful for drug delivery to both the upper and lower gastrointestinal tract are known.

Ion exchange resins have also been shown to prolong gastric retention, potentially by adhesion.

Gastric retention oral dosage forms may be used for delivery of drugs that are absorbed mainly from the upper gastrointestinal tract. For example, certain propofol prodrugs may exhibit limited colonic absorption, and be absorbed primarily from the upper gastrointestinal tract. Thus, dosage forms that release a propofol prodrug in the upper gastrointestinal tract and/or retard transit of the dosage form through the upper gastrointestinal tract will tend to enhance the oral bioavailability of the propofol prodrug or propofol metabolite.

Polymer matrices have also been used to achieve controlled release of drug over a prolonged period of time. Sustained or controlled release may be achieved by limiting the rate by which the surrounding gastric fluid can diffuse through the matrix and reach the drug, dissolve the drug and diffuse out again with the dissolved drug, or by using a matrix that slowly erodes, continuously exposing fresh drug to the surrounding fluid.

Other drug delivery devices that remain in the stomach for extended periods of time include, for example, hydrogel reservoirs containing particles, swellable hydroxypropylmethylcellulose polymers, planar bioerodible polymers, polymers comprising a plurality of compressible retention arms, hydrophilic water-swellable, cross-linked polymer particles, and albumin-cross-linked polyvinylpyrrolidone hydrogels.

In certain embodiments, propofol prodrugs may be practiced with a number of different dosage forms adapted to provide sustained release of a propofol prodrug upon oral administration. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In diffusion-controlled systems, water-insoluble polymers control the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate can be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of a drug is controlled by slowly soluble polymers or by microencapsulation. Once the coating is dissolved, the drug becomes available for dissolution. By varying the thickness and/or the composition of the coating or coatings, the rate of drug release can be controlled. In some dissolution-controlled systems, a fraction of the total dose may comprise an immediate-release component. Dissolution-controlled systems include encapsulated/reservoir dissolution systems and matrix dissolution systems. Encapsulated dissolution systems may be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices may be prepared, for example, by compressing a drug with a slowly soluble polymer carrier into a tablet form.

The rate of release of drug from osmotic pump systems is determined by the inflow of fluid across a semi-permeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and may be controlled within limits yielding relatively constant blood concentrations of the drug. Osmotic pump systems may provide a constant release of drug independent of the environment of the gastrointestinal tract. The rate of drug release may be modified by altering the osmotic agent and the size of the one or more orifices.

Release of drug from erosion-controlled systems is determined by the erosion rate of a carrier polymer matrix. Drug is dispersed throughout the polymer matrix and the rate of drug release depends on the erosion rate of the polymer. The drug-containing polymer may degrade from the bulk and/or from the surface of the dosage form.

Sustained release oral dosage forms may be in any appropriate form suitable for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

Sustained release oral dosage forms may release a propofol prodrug from the dosage form to facilitate the ability of the propofol prodrug and/or propofol metabolite to be absorbed from an appropriate region of the gastrointestinal tract, for example, in the small intestine, or in the colon. In certain embodiments, sustained release oral dosage forms may release a propofol prodrug from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms may release a propofol prodrug from the dosage form in a delivery pattern in which from about 0 wt % to about 20 wt % of the propofol prodrug is released in about 0 to about 4 hours, about 20 wt % to about 50 wt % of the propofol prodrug is released in about 0 to about 8 hours, about 55 wt % to about 85 wt % of the propofol prodrug is released in about 0 to about 14 hours, and about 80 wt % to about 100 wt % of the propofol prodrug is released in about 0 to about 24 hours. In certain embodiments, sustained release oral dosage forms may release a propofol prodrug from the dosage form in a delivery pattern in which from about 0 wt % to about 20 wt % of the propofol prodrug is released in about 0 to about 4 hours, about 20 wt % to about 50 wt % of the propofol prodrug is released in about 0 to about 8 hours, about 55 wt % to about 85 wt % of the propofol prodrug is released in about 0 to about 14 hours, and about 80 wt % to about 100 wt % of the propofol prodrug is released in about 0 to about 20 hours. In certain embodiments, sustained release oral dosage forms may release a propofol prodrug from the dosage form in a delivery pattern in which from about 0 wt % to about 20 wt % of the propofol prodrug is released in about 0 to about 2 hours, about 20 wt % to about 50 wt % of the propofol prodrug is released in about 0 to about 4 hours, about 55 wt % to about 85 wt % of the propofol prodrug is released in about 0 to about 7 hours, and about 80 wt % to about 100 wt % of the propofol prodrug is released in about 0 to about 8 hours.

Regardless of the specific form of oral dosage form used, a propofol prodrug may be released from the orally administered dosage form over a sufficient period of time to provide prolonged therapeutic concentrations of propofol in blood of a patient. Following oral administration, dosage forms comprising a propofol prodrug may provide a therapeutically effective concentration of propofol in the blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous period of time during which a therapeutically effective blood concentration of propofol is maintained may begin shortly after oral administration or following a time interval.

In certain embodiments, it may be desirable that the blood concentration of propofol be maintained at a level between a concentration that causes moderate sedation in the patient and a minimum therapeutically effective concentration for treating neuropathic pain for a continuous period of time. The blood concentration of propofol that causes moderate sedation (or anesthesia) in a patient can vary depending on the individual patient. Generally, a blood propofol concentration from about 1,500 ng/mL to about 2,000 ng/mL will produce moderate sedation, while a blood propofol concentration from about 3,000 ng/mL to about 10,000 ng/mL is sufficient to maintain general anesthesia. In certain embodiments, a minimum therapeutically effective blood propofol concentration will be about 10 ng/mL, about 20 ng/mL, about 50 ng/mL, about 100 ng/mL, about 100 ng/mL, about 200 ng/mL, about 400 ng/mL, or about 600 ng/mL. In certain embodiments, a therapeutically effective blood concentration of propofol for treating neuropathic pain is from about 10 ng/mL to less than about 5,000 ng/mL, and in certain embodiments from about 10 ng/mL to less than about 2,000 ng/mL. In certain embodiments, a therapeutically effective blood concentration of propofol for treating neuropathic pain is from about 10 ng/mL to less than a sedative concentration. In certain embodiments, a therapeutically effective blood concentration of propofol for treating neuropathic pain is from about 200 ng/mL to about 1,000 ng/mL. In certain embodiments, methods provided by the present disclosure provide a blood propofol concentration that, following oral administration to a patient, does not produce sedation and/or anesthesia in the patient. In certain embodiments, methods provided by the present disclosure provide a blood propofol concentration that, following oral administration to a patient, produces moderate sedation in a patient.

A therapeutically effective propofol blood concentration for treating neuropathic pain in a patient can also be defined in terms of the plasma concentration or pharmacokinetic profile. Thus, in certain embodiments, following oral administration of a dosage form comprising a propofol prodrug to a patient, the maximum propofol blood concentration, Cₘₐₓ, is less than that which causes sedation, for example, is less than about 1,500 ng/mL to about 2,000 ng/mL. In certain embodiments, following oral administration of a dosage form comprising a propofol prodrug to a patient, the propofol blood AUC during a 4-hour period may range from about 800 ng·h/mL to about 3,200 ng·h/mL and not cause sedation at any time following oral administration. In certain embodiments, following oral administration of a dosage form comprising a propofol prodrug to a patient, the propofol blood AUC during an 8-hour period may range from about 1,600 ng·h/mL to about 6,400 ng·h/mL and not cause sedation at any time following oral administration. In certain embodiments, following oral administration of a dosage form comprising a propofol prodrug to a patient, the propofol blood AUC during a 12-hour period may range from about 2,400 ng·h/mL to about 9,200 ng·h/mL and not cause sedation at any time following oral administration. In certain embodiments, following oral administration of a dosage form comprising a propofol prodrug to a patient, the propofol blood AUC during a 16-hour period may range from about 3,200 ng·h/mL to about 12,800 ng·h/mL and not cause sedation at any time following oral administration. In certain embodiments, following oral administration of a dosage form comprising a propofol prodrug to a patient, the propofol blood AUC during a 32-hour period may range from about 4,000 ng·h/mL to about 16,000 ng·h/mL and not cause sedation at any time following oral administration.

Propofol prodrugs may be absorbed from the gastrointestinal tract and enter the systemic circulation intact. In certain embodiments, a propofol prodrug exhibits an oral bioavailability of the propofol prodrug greater than about 40% that of an equivalent intravenous dose of the propofol prodrug, greater than about 60%, and in certain embodiments greater than about 80%. In certain of the foregoing embodiments, a propofol prodrug exhibits an oral bioavailability of propofol greater than about 10% that of an equivalent intravenous dose of propofol, greater than about 20%, greater than about 40% and in certain embodiments greater than about 60%.

### Methods of Use

Propofol prodrugs that provide a high oral bioavailability of propofol and dosage forms comprising such propofol prodrug may be used to treat neuropathic pain. Methods provided by the present disclosure comprise treating neuropathic pain in a patient by administering to a patient in need of such treatment a therapeutically effective amount of at least one propofol prodrug that provides a high oral bioavailability of propofol.

Propofol prodrugs provided by the present disclosure can be used to treat neuropathic pain. In certain embodiments, propofol prodrugs provided by the present disclosure can be used to treat neuropathic pain including post-herpetic neuralgia, peripheral neuropathy, trigeminal neuralgia, lower back pain, painful diabetic neuropathy, HIV-related neuropathic pain, cancer-related pain, or fibromyalgia.

The International Association for the Study of Neuropathic Pain defines neuropathic pain states as disorders that are characterized by lesions or dysfunction of the neural system(s) that under normal conditions transmit noxious information to the central nervous system. The mechanisms underlying neuropathic pain conditions are highly heterogeneous, however, all types of neuropathic pain are presumed to involve nerve injury and certain common aberrations in somatosensory processing in the central and/or peripheral nervous system. Potential causes of neuropathic pain, including physical damage, infection, and chemical exposure. NP can be generally classified as a focal/multifocal lesion of the peripheral nervous system, e.g., post-herpetic neuralgia, a generalized lesion of the peripheral nervous system, e.g., painful diabetic neuropathy, HIV-related NP), a lesion of the central nervous system, or a more complex neuropathic disorder. Peripheral neuropathic pain can arise as a consequence of trauma and surgery related nerve injury, e.g., brachial plexus injury; entrapment neuropathies such as lumbar disc compression, carpal tunnel syndrome; disease-related neuropathies, e.g., diabetes and HIV-AIDS; radiculopathy; complex regional pain syndrome; and tumor growth leading to nerve compression or infiltration. Central neuropathic pain can be the result of stroke, multiple sclerosis, and post-ischemic myelopathy; post-herpetic neuralgia; and post-traumatic spinal cord injury.

An essential part of neuropathic pain is a loss (partial or complete) of afferent sensory function and the paradoxical presence of certain hyperphenomena in the painful area. The nerve tissue lesion may be found in the brain, spinal cord, or the peripheral nervous system. Symptoms vary depending on the condition and can manifest as hyperalgesia (the lowering of pain threshold and an increased response to noxious stimuli), allodynia (the evocation of pain by non-noxious stimuli such as cold, warmth, or touch), hyperpathia (an explosive pain response that is suddenly evoked from cutaneous areas with increased sensory detection threshold when the stimulus intensity exceeds sensory threshold), paroxysms (a type of evoked pain characterized by shooting, electric, shock-like or stabbing pain that occur spontaneously, or following stimulation by an innocuous tactile stimulus or by a blunt pressure), paraesthesia (abnormal but non-painful sensations, which can be spontaneous or evoked, often described as pins and needles), dysesthesia (abnormal unpleasant but not necessarily painful sensation, which can be spontaneous or provoked by external stimuli), referred pain and abnormal pain radiation (abnormal spread of pain), and wind-up like pain and after-sensations (the persistence of pain long after termination of a painful stimulus).

Patients with neuropathic pain typically describe burning, lancinating, stabbing, cramping, aching, and sometimes vice-like pain. The pain can be paroxysmal or constant. Pathological changes to the peripheral nerve(s), spinal cord, and brain have been implicated in the induction and maintenance of chronic pain. Patients suffering from neuropathic pain typically endure chronic, debilitating episodes that are refractory to current pharmacotherapies and profoundly affect their quality of life. Currently available treatments for neuropathic pain, including tricyclic antidepressants and gabapentin, typically show limited efficacy in the majority of patients (Sindrup and Jensen, Pain 1999, 83, 389-400).

There are several types of neuropathic pain. A classification that relates to the type of damage or related pathophysiology causing a painful neuropathy includes neuropathies associated with mechanical nerve injury such as carpal tunnel syndrome, vertebral disk herniation, entrapment neuropathies, ulnar neuropathy, and neurogenetic thoracic outlet syndrome; metabolic disease associated neuropathies such as diabetic polyneuropathy; neuropathies associated with neurotropic viral disease such as herpes zoster and human immunodeficiency virus (HIV) disease; neuropathies associated with neurotoxicity such as chemotherapy of cancer or tuberculosis, radiation therapy, drug-induced neuropathy, and alcoholic neuropathy; neuropathies associated with inflammatory and/or immunologic mechanisms such as multiple sclerosis, anti-sulfatide antibody neuropathies, neuropathy associated with monoclonal gammopathy, Sjogren's disease, lupus, vasculitic neuropathy, polyclonal inflammatory neuropathies, Guillain-Barre syndrome, chronic inflammatory demyelinating neuropathy, multifocal motor neuropathy, paraneoplastic autonomic neuropathy, ganglinoic acetylcholine receptor antibody autonomic neuropathy, Lambert-Eaton myasthenic syndrome and myasthenia gravis; neuropathies associated with nervous system focal ischemia such as thalamic syndrome (anesthesia dolorosa); neuropathies associated with multiple neurotransmitter system dysfunction such as complex regional pain syndrome (CRPS); neuropathies associated with chronic/neuropathic pain such as osteoarthritis, lower back pain, fibromyalgia, cancer bone pain, chronic stump pain, phantom limb pain, and paraneoplastic neuropathies; toxic neuropathies (e.g., exposure to chemicals such as exposure to acrylamide, 3-chlorophene, carbamates, carbon disulfide, ethylene oxide, n-hexane, methyl n-butylketone, methyl bromide, organophosphates, polychlorinated biphenyls, pyriminil, trichlorethylene, or dichloroacetylene), focal traumatic neuropathies includingphantom and stump pain, monoradiculopathy, and trigeminal neuralgia; and central neuropathies including ischemic cerebrovascular injury (stroke), multiple sclerosis, spinal cord injury, Parkinson's disease, amyotrophic lateral sclerosis, syringomyelia, neoplasms, arachnoiditis, and post-operative pain; mixed neuropathies such as diabetic neuropathies including symmetric polyneuropathies such as sensory or sensorimotor polyneuropathy, selective small-fiber polyneuropathy, and autonomic neuropathy; focal and multifocal neuropathies such as cranial neuropathy, limb mononeuropathy, trunk mononeuro-pathy, mononeuropathy multiplex, and asymmetric lower limb motor neuropathy; and sympathetically maintained pain. Other neuropathies include focal neuropathy, glosopharyngeal neuralgia, ischemic pain, trigeminal neuralgia, atypical facial pain associated with Fabry's disease, Celiac disease, hereditary sensory neuropathy, or B₁₂-deficiency; mono-neuropathies, polyneuropathies, hereditary peripheral neuropathies such as Carcot-Marie-Tooth disease, Refsum's disease, Strumpell-Lorrain disease, and retinitis pigmentosa; acute polyradiculoneuropathy; and chronic polyradiculoneuropathy. Paraneoplastic neuropathies include paraneoplastic subacute sensory neuropathy, paraneoplastic motor neuron disease, paraneoplastic neuromyotonia, paraneoplastic demyelinating neuropathies, paraneoplastic vasculitic neuropathy, and paraneoplastic autonomic insufficiency.

Propofol prodrugs provided by the present disclosure can be used to treat any of the foregoing types of neuropathic pain.

The role of N-methyl-D-aspartate (NMDA) receptors in the development and maintenance of chronic pain associated with central and peripheral nerve injury is well documented. Consequently, NMDA antagonists have been proposed as potential therapeutics for neuropathic pain (Chizh and Headley, Curr Pharm Des 2005, 11(23), 2977-94). NMDA antagonists of different classes have shown efficacy in preclinical models as well as in patients with chronic pain, including neuropathic pain. However, a large-scale clinical use of NMDA antagonists for the treatment of neuropathic pain is limited by unacceptable side effects such as hallucinations, sedation, and ataxia), of currently available compounds of this class. In clinical studies, long-lasting relief in some neuropathic pain patients treated with NMDA antagonists has been observed (Pud et al., Pain 1998, 75(2-3), 349-54; Eisenberg et al., J Pain 2007, 8(3), 223-9; Rabben et al., J Pharmacol Exp Ther 1999, 289(2), 1060-1066; and Harbut et al., US 2005/0148673).

Experiments using animal models and limited clinical studies suggest that propofol may be useful in treating neuropathic pain. Locally injected propofol produces an antinociceptive effect in animal models of inflammatory pain (Guindon et al., Anesth Analg 2007, 104, 1563-1569). Propofol also is shown to be effective in treating neuropathic pain (complex regional pain syndrome and post-herpetic neuralgia) in individual patients (Harbut et al., US 2005/0148673) and to control cancer pain (Hooke et al., J Ped Oncology Nursing 2007, 24(1), 29-34).

The efficacy of propofol prodrugs provided by the present disclosure for treating one or more types of neuropathic pain can bee assessed in animal models of neuropathic pain and in clinical trials (Beggs and Salter, Drug Dev Res 2006, 67, 829-301). Animal models of peripheral nerve injury by ligation or transection include dorsal rhizotomy, spinal nerve ligation, sciatic nerve transaction, sciatic nerve cuff, partial nerve ligation, chronic constriction, and spared nerve injury. Other animal models of neuropathies involving immune system activation, and metabolic and chemically induced neuropathies include sciatic cyroneurolysis, HIV gp120, photochemical ischemia, anti-ganglioside antibody, streptozotocin-neuropathy, DDI-induced myelinopathy, vincristine, paclitaxel, and cisplatin.

The efficacy of propofol prodrugs provided by the present disclosure for treating various types of neuropathic pain can also be assessed in clinical trials using, for example, randomized double-blind placebo controlled methods. End points used in clinical trials for neuropathic pain can be determined using validated neuropathic pain criteria such as the Brief Pain Inventory, Categorical Scale, Gracety Pain Scale, Likert Scale, Neuropathic Pain Scale, Numerical Pain Scale, Short Form McGill Pain Questionnaire, Verbal Pain Scale, Visual Analog Scale (VAS), VAS Pain Intensity Scale, and/or VAS Pain Relief Scale.

### Dose

The amount of a propofol prodrug that will be effective in the treatment of a particular disease, disorder, or condition disclosed herein will depend on the nature of the disease, disorder, or condition, and can be determined by standard clinical techniques known in the art. In addition, in vitro or in vivo assays may optionally be employed to help identify optimal dosage ranges. The amount of a compound administered can depend on, among other factors, the patient being treated, the weight of the patient, the health of the patient, the disease being treated, the severity of the affliction, the route of administration, the potency of the compound, and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

In certain embodiments, a therapeutically effective dose of a propofol prodrug may comprise from about 1 mg-equivalents to about 2,000 mg-equivalents of propofol per day, from about 5 mg-equivalents to about 1000 mg-equivalents of propofol per day, and in certain embodiments, from about 10 mg-equivalents to about 500 mg-equivalents of propofol per day.

A dose may be administered in a single dosage form or in multiple dosage forms. When multiple dosage forms are used the amount of a propofol prodrug contained within each of the multiple dosage forms may be the same or different.

In certain embodiments, an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a highly orally bioavailable propofol prodrug maybe within a range of circulating concentrations in for example the blood, plasma, or central nervous system, that is therapeutically effective, that is less than a sedative dose, and that exhibits little or no toxicity. A dose may vary within this range depending upon the dosage form employed.

During treatment a dose and dosing schedule may provide sufficient or steady state systemic concentrations of a therapeutically effective amount of propofol to treat a disease. In certain embodiments, an escalating dose may be administered.

Propofol prodrugs that provide a high oral bioavailability of propofol may be administered orally, and may be administered at intervals for as long as necessary to obtain an intended or desired therapeutic effect.

### Combination Therapy

Propofol prodrugs that provide a high oral bioavailability propofol may be used in combination therapy with at least one other therapeutic agent. Propofol prodrugs and another therapeutic agent(s) can act additively or, and in certain embodiments, synergistically. In some embodiments, propofol prodrugs may be administered concurrently with the administration of another therapeutic agent, such as for example, a compound for treating neuropathic pain. In some embodiments, a propofol prodrug may be administered prior or subsequent to administration of another therapeutic agent, such as for example, a compound for treating neuropathic pain.

Methods provided by the present disclosure include administering one or more propofol prodrugs and one or more other therapeutic agents provided that the combined administration does not inhibit the therapeutic efficacy of the one or more propofol prodrugs and/or other therapeutic agent and/or does not produce adverse combination effects.

In certain embodiments, propofol prodrugs may be administered concurrently with the administration of another therapeutic agent, which may be part of the same pharmaceutical composition or dosage form as or in a different composition or dosage form than that containing a propofol prodrug. When a propofol prodrug is administered concurrently with another therapeutic agent that potentially can produce adverse side effects including, but not limited to, toxicity, the therapeutic agent may be administered at a dose that falls below the threshold at which the adverse side effect is elicited.

In certain embodiments, propofol prodrugs may be administered prior or subsequent to administration of another therapeutic agent. In certain embodiments of combination therapy, the combination therapy comprises alternating between administering a propofol prodrug and a composition comprising another therapeutic agent, e.g., to minimize adverse side effects associated with a particular drug.

In certain embodiments, acamprosate prodrugs provided by the present disclosure and pharmaceutical compositions thereof may be administered to a patient for treating neuropathic pain in combination with a therapy or another therapeutic agent known or believed to be effective in treating neuropathic pain.

Examples of drugs useful for treating pain include opioid analgesics such as morphine, codeine, fentanyl, meperidine, methadone, propoxyphene, levorphanol, hydromorphone, oxycodone, oxymorphone, tramadol and pentazocine; non-opioid analgesics such as aspirin, ibuprofen, ketoprofen, naproxen, and acetaminophen; non-steroidal anti-inflammatory drugs such as aspirin, choline magnesium trisalicylate, diflunisal, salsalate, celecoxib, rofecoxib, valdecoxib, diclofenac, etodolac, fenoprofen, flubiprofen, ibuprofen, indomethacin, ketoprofen, ketorolac, meclofanamate, mefenamic acid, meloxicam, nabumetone, naproxen, oxaprozin, piroxicam, sulindac, and tometin; antiepileptics such as gabapentin, pregabalin, carbamazepine, phenyloin, lamotrigine, and topiramate; antidepressants such as duloxetine, amitriptyline, venlafaxine, nortryptyline, imipramine, and desipramine; local anesthetics such as lidocaine, and mexiletine; NMDA receptor antagonists such as dextropethorphan, memantine, and ketamine; N-type calcium-channel blockers such as ziconotide; vanilloid receptor-1 modulators such as capsaicin; cannabinoid receptor modulators such as sativex; neurokinin receptor antagonists such as lanepitant; other analgesics such as neurotropin; and other drugs such as desipramine, clonazepam, divalproex, oxcarbazepine, divalproex, butorphanol, valdecoxib, vicoprofen, pentazocine, propoxyhene, fenoprofen, piroxicam, indometnacin, hydroxyzine, buprenorphine, benzocaine, clonidine, flurbiprofen, meperidine, lacosamide, desvenlafaxine, and bicifadine.

Non-pharmacological therapies for treating neuropathic pain include transcutaneous electrical nerve stimulation, percutaneous electrical nerve stimulation, and acupuncture.

### EXAMPLES

The following examples describe in detail methods of using propofol prodrugs. It will be apparent to those skilled in the art that many modifications, both to materials and methods, may be practiced without departing from the scope of the disclosure.

### Example 1

### Pharmacokinetics of Compound (2) and Propofol in Rats

Propofol or compound (2) was administered as an intravenous bolus injection or by oral gavage to groups of four to six adult male Sprague-Dawley rats (about 250 g). Animals were conscious at the time of the experiment. When orally administered, propofol or compound (2) was administered as an aqueous solution at a dose equivalent to propofol per kg body weight. When administered intravenously, propofol was administered as a solution (Diprivan®, Astra-Zeneca) at a dose equivalent to 10 or 15 mg of propofol per kg body weight. Animals were fasted overnight before the study and for 4 hours post-dosing. Blood samples (0.3 mL) were obtained via a jugular vein cannula at intervals over 8 hours following oral dosing. Blood was quenched immediately using acetonitrile with 1% formic acid and then was frozen at −80° C. until analyzed.

Three hundred (300) μL of 0.1% formic acid in acetonitrile was added to blank 1.5 mL tubes. Rat blood (300 μL) was collected at different times into tubes containing EDTA and vortexed to mix. A fixed volume of blood (100 μL) was immediately added into the Eppendorf tube and vortexed to mix. Ten (10) μL of a propofol standard stock solution (0.04, 0.2, 1, 5, 25, and 100 μg/mL) was added to 90 μL of blank rat blood quenched with 300 μL of 0.1% formic acid in acetonitrile. Then, 20 μL of para-chlorophenylalanine was added to each tube to make a final calibration standard (0.004, 0.02, 0.1, 0.5, 2.5, and 10 μg/mL). Samples were vortexed and centrifuged at 14,000 rpm for 10 min. The supernatant was analyzed by LC/S/MS.

An API 4000 LC/MS/MS spectrometer equipped with Agilent 1100 binary pumps and a CTC HTS-PAL autosampler and a Phenomenex Synergihydro-RP 4.6×30 mm column were used in the analysis. The mobile phase for propofol analysis was (A) 2 mM ammonium acetate, and (B) 5 mM ammonium acetate in 95% acetonitrile. The mobile phase for the analysis of compound (2) was (A) 0.1% formic acid, and (B) 0.1% formic acid in acetonitrile. The gradient condition was: 10% B for 0.5 min, then to 95% B in 2.5 min, then maintained at 95% B for 1.5 min. The mobile phase was returned to 10% B for 2 min. An APCI source was used on the API 4000. The analysis was done in negative ion mode for propofol and in positive ion mode for compound (2). The MRM transition for each analyte was optimized using standard solutions. Five (5) μL of each sample was injected. Non-compartmental analysis was performed using WinNonlin (v.3.1 Professional Version, Pharsight Corporation, Mountain View, Calif.) on individual animal profiles. Summary statistics on major parameter estimates was performed for Cₘₐₓ (peak observed concentration following dosing), Tₘₐₓ (time to maximum concentration is the time at which the peak concentration was observed), AUC₀₋ₜ (area under the serum concentration-time curve from time zero to last collection time, estimated using the log-linear trapezoidal method), AUC_{(0-∞)}, (area under the serum concentration time curve from time zero to infinity, estimated using the log-linear trapezoidal method to the last collection time with extrapolation to infinity), and t_{1/2} (terminal half-life).

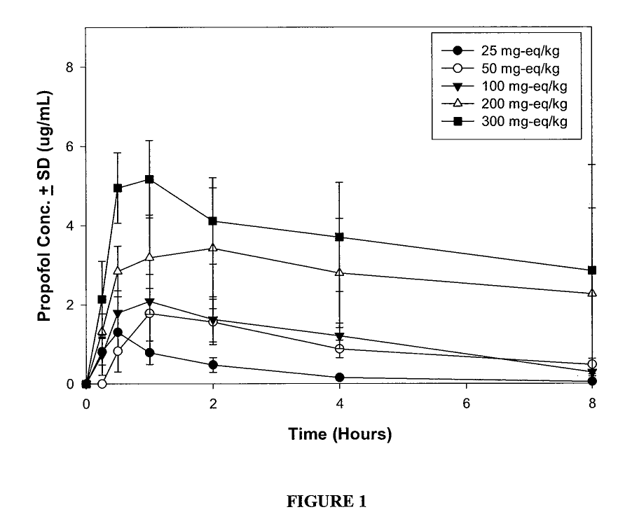

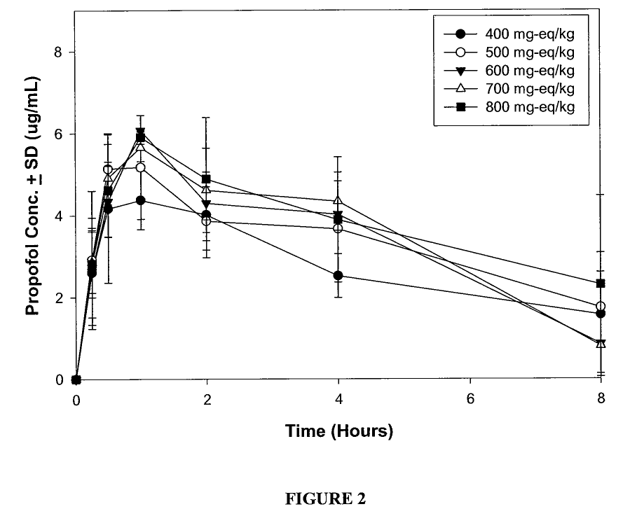

The oral bioavailability (F %) of propofol was determined by comparing the area under the propofol concentration vs time curve (AUC) following oral administration of compound (2) with the AUC of the propofol concentration vs time curve following intravenous administration of propofol on a dose normalized basis. The results from these studies are summarized in FIG. 1, FIG. 2, and Table 1.

TABLE 1
Pharmacokinetic Parameter Summary for Rat Study

| Compound | | | | | | |
|---|---|---|---|---|---|---|
| (2) | | | | | | |
| Dose Level | Cₘₐₓ | Tₘₐₓ | T_{1/2-1} | AUCₜ | AUC_{inf} | Fₚₒ |
| (mg-eq/kg) | (μg/mL) | (hr) | (hr) | (hr · μg/mL) | (hr · μg/mL) | (%) |
| 25 | 0.8 (0.2) | 1.7 (0.5) | 2.2 (0.5) | 2.5 (0.7) | 2.8 (0.7) | 65 (17) |
| 50 | 2.0 (0.8) | 2.0 (1.2) | 3.2 (2.9) | 5.0 (1.7) | 6.0 (2.0) | 78 (23) |
| 100 | 2.2 (0.4) | 1.1 (0.6) | 2.8 (0.7) | 9.1 (1.3) | 10.3 (0.9) | 61 (6) |
| 200 | 3.4 (2.0) | 1.3 (1.1) | 5.0 (4.3) | 18.5 (12.2) | 24.6 (6.7) | 72 (20) |
| 300 | 4.6 (0.7) | 0.8 (0.4) | 2.4 (0.4) | 18.7 (0.5) | 20.9 (0.1) | 41 (0) |
| 400 | 4.7 (0.7) | 1.0 (0.7) | 2.6 (0.8) | 22.0 (2.7) | 25.0 (1.2) | 37 (2) |
| 500 | 5.0 (0.6) | 2.3 (1.7) | 11.1 (0.1) | 41.7 (17.5) | 53.4 (23.9) | 83 (28) |
| 600 | 6.1 (0.0) | 1.0 (0.0) | 2.4 (0.0) | 25.4 (22.4) | 33.4 (11.2) | 33 (11) |
| 700 | 5.6 (0.3) | 1.0 (0.0) | 3.8 (2.7) | 24.3 (5.2) | 40.1 (18.7) | 39 (21) |
| 800 | 6.0 (0.5) | 1.3 (0.6) | 6.1 (5.7) | 29.5 (11.9) | 60.6 (53.6) | 60 (53) |

### Example 2

### Pharmacokinetics of Compound (2) and Propofol in Dogs

Compound (2) or propofol was administered by oral gavage or as an intravenous bolus injection, respectively, to groups of two to four adult male Beagle dogs (about 8 kg) as solutions in water. Animals were fasted overnight before the study and for 4 hours post-dosing. Blood samples (1.0 mL) were obtained via the femoral vein at intervals over 24 hours after oral dosing. Blood was quenched immediately using acetonitrile with 1% formic acid and then frozen at −80° C. until analyzed. Compound (2) was administered to dogs with a minimum of 7-day wash out period between dosing sessions.

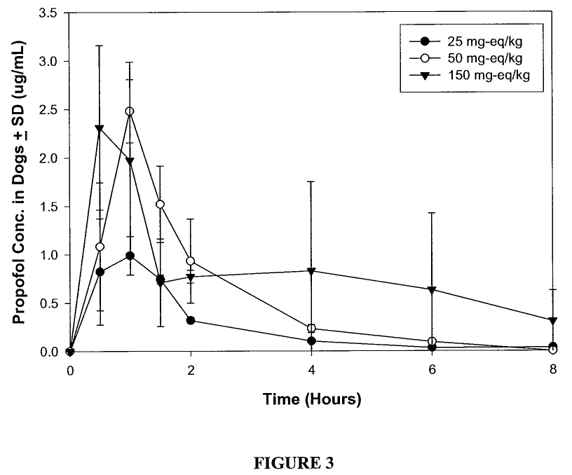

Blood sample preparation and LC/MS/MS analysis were the same as for the rat study described in Example 1. The pharmacokinetics of propofol following oral administration of compound (2) to dogs is summarized in FIG. 3 and Table 2.

TABLE 2
Pharmacokinetic Parameter Summary for Dog Study

| Compound | | | | | | |
|---|---|---|---|---|---|---|
| (2) | | | | | | |
| Dose Level | Cₘₐₓ | Tₘₐₓ | T_{1/2-1} | AUCₜ | AUC_{inf} | Fₚₒ |
| (mg-eq/kg) | (μg/mL) | (hr) | (hr) | (hr · μg/mL) | (hr · μg/mL) | (%) |
| 25 | 1.0 (0.3) | 0.8 (0.4) | 0.9 (0.1) | 1.8 (0.5) | 2.0 (0.5) | 37 (10) |
| 50 | 2.5 (0.3) | 1.0 (0.0) | 1.1 (0.1) | 4.3 (0.7) | 4.4 (0.7) | 41 (6) |
| 150 | 2.3 (0.8) | 0.5 (0.0) | 2.3 (0.6) | 6.7 (5.0) | 7.9 (6.5) | 25 (20) |

### Example 3

### Toxicity Studies

Acute toxicity studies in rats were undertaken to assess the tolerance of a single oral dose of compound (2) formulated in water. The results indicated that compound (2) was well tolerated at levels from about 49 mg-eq/kg to about 1552 mg-eq/kg of administered compound. Transient hypoactivity was observed at doses from about 49 mg-eq/kg up to about 388 mg-eq/kg within about 30 minutes of dose and maintained up to 4 hours post dose. Sedation was observed at doses from about 582 mg-eq/kg up to about 970 mg-eq/kg within about 1.5 hours of dose and lasted up to 4 hours post dose. Anesthesia was observed at doses from about 1164 mg-eq/kg up to about 1552 mg-eq/kg within about 1 hour of dose and lasted up to about 2 hours post dose. Complete recovery from hypoactivity, sedation, and anesthesia occurred in all rats within about 8 hours after dose. Doses above about 1552 mg-eq/kg (about 800 mg-eq/kg of propofol) were not tested.

Acute toxicity studies were also performed by orally administering a single dose of compound (2) formulated in water to groups of male beagle dogs at doses from about 25 mg-eq/kg to about 150 mg-eq/kg. Results indicated that at these doses compound (2) was well tolerated in dogs. No sedation or anesthesia was observed at these doses.

Multiple dose studies in rats were performed by orally administering compound (2) formulated in water to groups of male rats at doses of 49 mg-eq/kg to 97 mg-eq/kg for a period of five days, by oral gavages administered once a day. No adverse effects were observed in the multiple dose studies. Results indicated that compound (2) was well tolerated by rats. No sedation or anesthesia was observed at these doses.

### Description 1

### Use of Animal Models to Assess the Efficacy of Propofol Prodrugs for Treating Neuropathic Pain

### Inflammatory Pain—Formalin Test

A formalin assessment test is used. Fifty μL of a 5% formalin solution is injected subcutaneously into the dorsal aspect of the right hind paw and the rats are then individually placed into clear observation cages. Rats are observed for a continuous period of 60 min or for periods of time corresponding to phase I (from 0 to 10 min following formalin injection) and phase II (from 30 to 50 min following formalin injection) of the formalin test (Abbott et al., Pain 1995, 60, 91-102). The number of flinching behaviors of the injected paw is recorded using a sampling technique in which each animal is observed for one 60-sec period during each 5-min interval. Test compound is administered 30 min or other appropriate interval prior to formalin injection.

### Inflammatory Pain—Carrageenan-Induced Acute Thermal Hyperalgesia and Edema

Paw edema and acute thermal hyperalgesia are induced by injecting 100 μL of a 1% solution of λ-carrageenan in physiological saline into the plantar surface of the right hind paw. Thermal hyperalgesia is determined 2 h following carrageenan injection, using a thermal paw stimulator. Rats are placed into plastic cubicles mounted on a glass surface maintained at 30° C. and a thermal stimulus in the form of radiant heat emitted form a focused projection bulb is then applied to the plantar surface of each hind paw. The stimulus current is maintained at about 4.50 Amp, and the maximum time of exposure is set at about 20 sec to limit possible tissue damage. The elapsed time until a brisk withdrawal of the hind paw from the thermal stimulus is recorded automatically using photodiode motion sensors. The right and left hind paw of each rat is tested in three sequential trials at about 5-min intervals. Carrageenan-induced thermal hyperalgesia of paw withdrawal latency (PWLₜₕₑᵣₘₐₗ) is calculated as the mean of the two shortest latencies. Test compound is administered 30 min before assessment of thermal hyperalgesia.

The volume of paw edema is measured using water displacement with a plethysmometer 2 h following carrageenan injection by submerging the paw up to the ankle hairline (approx. 1.5 cm). The displacement of the volume is measured by a transducer and recorded. Test compound is administered at an appropriate time following carrageenan injection, such as for example, 30 min or 90 min.

### Visceral Pain

Thirty min following administration of test compound, mice receive an injection of 0.6% acetic acid in sterile water (10 mL/kg, i.p.). Mice are then placed in table-top Plexiglass observation cylinders (60 cm high×40 cm diameter) and the number of constrictions/writhes (a wave of mild constriction and elongation passing caudally along the abdominal wall, accompanied by a slight twisting of the trunk and followed by bilateral extension of the hind limbs) is recorded during the 5-20 min following acetic acid injection for a continuous observation period of 15 min.

### Neuropathic Pain—Spinal Nerve Ligation

Rats receive unilateral ligation of the lumbar 5 (L5) and lumbar 6 (L6) spinal nerves as described. The left L5 and L6 spinal nerves of the rat are isolated adjacent to the vertebral column and tightly ligated with a 5-0 silk suture distal to the dorsal root ganglia, and care is taken to avoid injury of the lumbar 4 (L4) spinal nerve. Control rats undergo the same procedure but without nerve ligation. All animals are allowed to recover for at least 1 week and not more than 3 weeks prior to assessment of mechanical allodynia. Mechanical allodynia is measure using calibrated von Frey filaments. Rats are placed into inverted plastic containers (20×12.5×20 cm) on top of a suspended wire mesh grid and acclimated to the test chamber for 20 min. The von Frey filaments are presented perpendicularly to the plantar surface of the selected hind paw, and then held in this position for approximately 8 s with enough force to cause a slight bend in the filament. Positive responses include an abrupt withdrawal of the hind paw from the stimulus or flinching behavior immediately following removal of the stimulus. A 50% paw withdrawal threshold (PWT) is determined. Rats with a PWT≦5.0 g are considered allodynic and utilized to test the analgesic activity of a test compound. The test compound can be administered 30 min prior to the assessment of mechanical allodynia.

### Neuropathic Pain—Chronic Constriction Injury of the Sciatic Nerve

A model of chronic constriction injury of the sciatic nerve-induced neuropathic pain according to the method of Bennett and Xie, Pain 1988, 33, 87-107, is used. The right common sciatic nerve is isolated at mid-thigh level and loosely ligated by four chromic gut (4-0) ties separated by an interval of 1 mm. Control rats undergo the same procedure but without sciatic nerve constriction. All animals are allowed to recover for at least 2 weeks and for no more than 5 weeks prior to testing of mechanical allodynia. Allodynic PWT is assessed in the animals as described for animals with spinal nerve ligation. Only rats with a PWT≦5.0 g are considered allodynic and utilized to evaluate the analgesic activity of a test compound. Test compound is administered 30 min or other appropriate time prior to the assessment of mechanical allodynia.

### Neuropathic Pain—Vincristine-Induced Mechanical Allodynia

A model of chemotherapy-induced neuropathic pain is produced by continuous intravenous vincristine infusion (Nozaki-Taguchi et al., Pain 2001, 93, 69-76). Anesthetized rats undergo a surgical procedure in which the jugular vein is catheterized and a vincristine-primed pump is implanted subcutaneously. Fourteen days of intravenous infusion of vincristine (30 μg/kg/day) results in systemic neuropathic pain of the animal. Control animals undergo the same surgical procedure, with physiological saline infusion. PWT of the left paw is assessed in the animals 14 days post-implantation as described for the spinal nerve ligation model. Test compound is administered 30 min prior to the test for mechanical allodynia in rats with PWT≦5.00 g before treatment.

### Post-Operative Pain

A model of post-operative pain is performed in rats as described by Brennan et al., Pain 1996, 64, 493-501. The plantar aspect of the left hind paw is exposed through a hole in a sterile plastic drape, and a 1-cm longitudinal incision is made through the skin and fascia, starting 0.5 cm from the proximal edge of the heel and extending towards the toes. The plantaris muscle is elevated and incised longitudinally leaving the muscle origin and insertion points intact. After hemostasis by application of gently pressure, the skin is apposed with two mattress sutures using 5-0 nylon. Animals are then allowed to recover for 2 h following surgery, at which time mechanical allodynia and thermal hyperalgesia are assessed.

Effects of test compound on mechanical allodynia are assessed 30 min following administration, with PWT being examined in these animals for both the injured and non-injured paw as described for the spinal nerve ligation model with the von Frey filament systematically pointing towards the medial side of the incision. In a separate experiment, the effects of test compound on thermal hyperalgesia are assessed 30 min following administration of test compound, with PWLₜₕₑᵣₘₐₗ being determined as described for the carrageen-induced thermal hyperalgesia model with the thermal stimulus applied to the center of the incision of the paw planter aspect.

Finally, it should be noted that there are alternative ways of implementing the disclosures contained herein. Accordingly, the present embodiments are to be considered as illustrative and not restrictive, and the claims are not to be limited to the details given herein, but may be modified within the scope and equivalents thereof.

1. A method of treating neuropathic pain in a patient comprising orally administering to a patient in need of such treatment a therapeutically effective amount of a propofol prodrug of Formula (I): 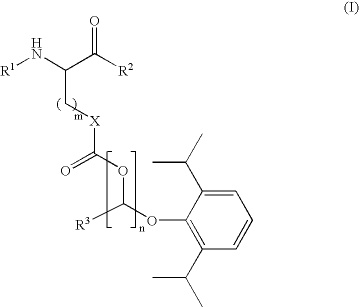
or a pharmaceutically acceptable salt thereof, wherein:
X is chosen from a bond, —CH₂—, —NR¹¹—, —O—, and —S—;
m is chosen from 1 and 2;
n is chosen from 0 and 1;
R¹ is chosen from hydrogen, [R⁵NH(CHR⁴)ₚC(O)]—, R⁶—, R⁶C(O)—, and R⁶OC(O)—;
R² is chosen from —OR⁷ and —[NR⁸(CHR⁹)_{q}C(O)OR^{7];}
p and q are independently chosen from 1 and 2;
each R³ is independently chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;
each R⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁴ and R⁵ are attached to adjacent atoms then R⁴ and R⁵ together with the atoms to which they are bonded form a ring chosen from a heterocycloalkyl and substituted heterocycloalkyl ring;
R⁵ is chosen from hydrogen, R⁶—, R⁶C(O)—, and R⁶OC(O)—;
R⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
each R⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁸ and R⁹ are attached to adjacent atoms then R⁸ and R⁹ together with the atoms to which they are bonded form a ring chosen from a heterocycloalkyl and substituted heterocycloalkyl ring; and
R¹¹ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
with the provisos that:
when R¹ is [R⁵NH(CHR⁴)ₚC(O)]— then R² is —OR⁷; and
when R² is —[NR⁸(CHR⁹)_{q}C(O)OR⁷] then R¹ is not [R⁵NH(CHR⁴)PC(O)]—.

2. The method of claim 1, wherein the propofol prodrug is (S)-2-amino-3-(2,6-diisopropylphenoxycarbonyloxy)-propanoic acid or a pharmaceutically acceptable salt thereof.

3. The method of claim 2, wherein the pharmaceutically acceptable salt of (S)-2-amino-3-(2,6-diisopropylphenoxycarbonyloxy)-propanoic acid is the mesylate salt.

4. The method of claim 3, wherein the salt is crystalline 2-amino-3-(2,6-diisopropylphenoxycarbonyloxy)-propanoic acid mesylate.

5. A method of treating neuropathic pain in a patient comprising orally administering to a patient in need of such treatment a therapeutically effective amount of a propofol prodrug of Formula (II): 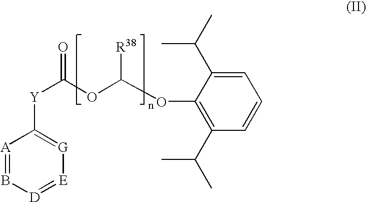
or a pharmaceutically acceptable salt thereof, wherein:
n is chosen from 0 and 1;
Y is chosen from a bond, CR²¹R²², NR²³, O, and S;
A is chosen from CR²⁴ and N;
B is chosen from CR²⁵ and N;
D is chosen from CR²⁶ and N;
E is chosen from CR²⁷ and N;
G is chosen from CR²⁸ and N;
R³⁸ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;
R²¹ and R²² are independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
R²³ is chosen from hydrogen, alkyl, substituted alkyl, aryl, arylalkyl, cycloalkyl, and heteroaryl;
R²⁴ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁵ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁶ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁷ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁸ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
W is chosen from a bond, —CR³²R³³—NR³⁴, O, and S;
Z is chosen from —CR³⁵R³⁶—NR³⁷, O, and S;
k is chosen from 0 and 1;
r is chosen from 1, 2, and 3;
each of R²⁹, R³⁰, R³¹, R³², R³³, R³⁵ and R³⁶ is independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and
R³⁴ and R³⁷ are independently chosen from hydrogen, alkyl, substituted alkyl, aryl, arylalkyl, cycloalkyl, and heteroaryl;
with the provisos that:
at least one of A, B, D, E, and G is not N;
one and only one of R²⁴, R²⁵, R²⁶, R²⁷, or R²⁸ is —W[C(O)]ₖZ(CR²⁹R³⁰)ᵣCO₂R³¹; and
if k is 0 then W is a bond.

6. A method of treating neuropathic pain in a patient comprising orally administering to a patient in need of such treatment a therapeutically effective amount of a propofol prodrug of Formula (III): 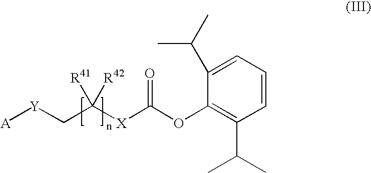
or a pharmaceutically acceptable salt thereof, wherein:
each R⁴¹ and R⁴² is independently chosen from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; or R⁴¹ and R⁴² together with the carbon atom to which they are bonded form a ring chosen from a cycloalkyl, substituted cycloalkyl, heterocycloalkyl, and substituted heterocycloalkyl ring;
A is chosen from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; or A, Y, and one of R⁴¹ and R⁴² together with the atoms to which they are bonded form a ring chosen from a heterocycloalkyl and substituted heterocycloalkyl ring;
Y is chosen from —O— and —NR⁴³—;
R⁴³ is chosen from hydrogen, alkyl, substituted alkyl, arylalkyl, and substituted arylalkyl;
n is an integer from 1 to 5;
X is chosen from —NR⁴⁴—, —O—, —CH₂, and —S—; and
R⁴⁴ is chosen from hydrogen, alkyl, substituted alkyl, arylalkyl, and substituted arylalkyl.

7. A method of treating neuropathic pain in a patient comprising orally administering to a patient in need of such treatment a therapeutically effective amount of a propofol prodrug of Formula (IV): 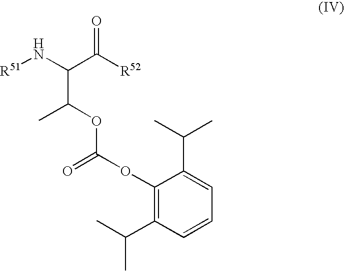
or a pharmaceutically acceptable salt thereof, wherein:
R⁵¹ is chosen from hydrogen, [R⁵⁵NH(CHR⁵⁴)ₚC(O)]—, R⁵⁶—, R⁵⁶C(O), and R⁵⁶OC(O)—;
R⁵² is chosen from —OR⁵⁷ and —[NR⁵⁸(CHR⁵⁹)_{q}C(O)OR⁵⁷];
p and q are independently chosen from 1 and 2;
each R⁵⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁵⁴ and R⁵⁵ are bonded to adjacent atoms then R⁵⁴ and R⁵⁵ together with the atoms to which they are bonded form a ring chosen from a heterocycloalkyl and substituted heterocycloalkyl ring;
R⁵⁵ is chosen from hydrogen, R⁵⁶—, R⁵⁶C(O)—, and R⁵⁶OC(O)—;
R⁵⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁵⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁵⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and
each R⁵⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl; or when R⁵⁸ and R⁵⁹ are bonded to adjacent atoms then R⁵⁸ and R⁵⁹ together with the atoms to which they are bonded form a ring chosen from a heterocycloalkyl and substituted heterocycloalkyl ring;
with the proviso that when R⁵² is —[NR⁵⁸(CHR⁵⁹)_{q}C(O)OR⁵⁷] then R⁵¹ is not [R⁵⁵NH(CHR⁵⁴)ₚC(O)]—.

8. The method of claim 7, wherein the propofol prodrug is 2-amino-3-methyl-3-(2,6-diisopropyl-phenoxycarbonyloxy)-propanoic acid or a pharmaceutically acceptable salt thereof.

9. The method of claims 1, comprising maintaining a propofol concentration in the blood of the patient from 10 ng/mL to 2,000 ng/mL for at least about 4 hours following oral administration of the propofol prodrug to the patient.

10. The method of claim 1, wherein the therapeutically effective amount is less than an amount that causes moderate sedation in the patient.

11. The method of claim 1, wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

12. The method of claim 1, wherein the propofol prodrug is administered as a sustained release oral formulation.

13. The method of claims 4, comprising maintaining a propofol concentration in the blood of the patient from 10 ng/mL to 2,000 ng/mL for at least about 4 hours following oral administration of the propofol prodrug to the patient.

14. The method of claim 4, wherein the therapeutically effective amount is less than an amount that causes moderate sedation in the patient.

15. The method of claim 4, wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

16. The method of claim 4, wherein the propofol prodrug is administered as a sustained release oral formulation.

17. The method of claim 5, comprising maintaining a propofol concentration in the blood of the patient from 10 ng/mL to 2,000 ng/mL for at least about 4 hours following oral administration of the propofol prodrug to the patient.

18. The method of claim 5, wherein the therapeutically effective amount is less than an amount that causes moderate sedation in the patient.

19. The method of claim 5, wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

20. The method of claim 5, wherein the propofol prodrug is administered as a sustained release oral formulation.

21. The method of claim 6, comprising maintaining a propofol concentration in the blood of the patient from 10 ng/mL to 2,000 ng/mL for at least about 4 hours following oral administration of the propofol prodrug to the patient.

22. The method of claim 6, wherein the therapeutically effective amount is less than an amount that causes moderate sedation in the patient.

23. The method of claim 6, wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

24. The method of claim 6, wherein the propofol prodrug is administered as a sustained release oral formulation.

25. The method of claim 7, comprising maintaining a propofol concentration in the blood of the patient from 10 ng/mL to 2,000 ng/mL for at least about 4 hours following oral administration of the propofol prodrug to the patient.

26. The method of claim 7, wherein the therapeutically effective amount is less than an amount that causes moderate sedation in the patient.

27. The method of claim 7, wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

28. The method of claim 7, wherein the propofol prodrug is administered as a sustained release oral formulation.
